SEQUENCE LISTING (6 sequences)

SEQ ID NO: 1 - DNA, 20 bp, Artificial Sequence
  synthetic construct; gRNA binding site
ttggatggtgcttcaagtta

SEQ ID NO: 2 - DNA, 20 bp, Artificial Sequence
  synthetic construct, gRNA binding site
ctacaagggactttccgctg

SEQ ID NO: 3 - DNA, 20 bp, Artificial Sequence
  synthetic construct; gRNA binding site
tctacaagggactttccgct

SEQ ID NO: 4 - DNA, 20 bp, Artificial Sequence
  synthetic construct; reverse primer
acaggccaggattaactgcg

SEQ ID NO: 5 - DNA, 22 bp, Artificial Sequence
  synthetic construct; forward primer
ggaagggctaattcactcccaa

SEQ ID NO: 6 - DNA, 30 bp, Artificial Sequence
  synthetic construct; hairpin adapter
  misc_feature (29)..(30): phosphorothioate linkage between position 29 and 30
cggtggaccgatgatcuatcggtccaccgt

(19) United States
(12) Patent Application Publication
(10) Pub. No.: US 2023/0313193 A1
(43) Pub. Date: Oct. 5, 2023

(54) METHODS AND COMPOSITIONS FOR CRISPR/CAS9 GUIDE RNA EFFICIENCY AND SPECIFICITY AGAINST GENETICALLY DIVERSE HIV-1 ISOLATES

(71) Applicants: United States Government As Represented By The Department of Veterans Affairs, Washington, DC (US); Trustees Of Dartmouth College, Hanover, NH (US)

(72) Inventors: Alexandra L. Howell, Lyme, NH (US); Susan K. Eszterhas, Plainfield, NH (US); Matthew S. Hayden, Hanover, NH (US)

(21) Appl. No.: 18/016,109

(22) PCT Filed: Jul. 13, 2021
(86) PCT No.: PCT/US2021/041385

§ 371 Date: Jan. 13, 2023

Related U.S. Application Data
(60) Provisional application No. 63/051,212, filed on Jul. 13, 2020.

(51) Int. Cl. C12N 15/113; C12N 15/86

(52) U.S. Cl. CPC C12N 15/1132; C12N 15/86; C12N 2740/15041; C12N 2310/20

(57) ABSTRACT

Disclosed are guide RNAs (gRNAs) that specifically bind the 5′ LTR human immunodeficiency virus-1 (HIV-1) sequence comprising TTGGATGGTGCTTCAAGTTA (SEQ ID NO: 1). Disclosed are gRNAs that specifically bind the 5′ LTR HIV-1 sequence comprising CTACAAGGGACTTTCCGCTG (SEQ ID NO: 2). Disclosed are gRNAs that specifically bind the 5′ LTR HIV-1 sequence comprising TCTACAAGGGACTTTCCGCT (SEQ ID NO: 3). Disclosed are nucleic acid sequences comprising a nucleic acid sequence encoding one or more gRNAs, wherein said one or more gRNAs hybridize with a target sequence in HIV-1, wherein the target sequence is selected from the group consisting of SEQ ID NO: 1, SEQ ID NO: 2, and SEQ ID NO: 3. Disclosed are vectors comprising a nucleic acid sequence encoding one or more gRNAs, wherein the one or more gRNA hybridizes with a target sequence in HIV-1, wherein the target sequence is selected from the group consisting of SEQ ID NO: 1, SEQ ID NO: 2, and SEQ ID NO: 3. Disclosed are methods for inhibiting the function of a target HIV-1 DNA sequence in a cell or removing a target HIV-1 DNA sequence from a cellular genome comprising contacting a cell comprising a cellular genome and harboring a HIV-1 genome comprising a target HIV-1 DNA sequence integrated into the cellular genome with one or more gRNAs, or nucleic acids encoding said one or more gRNAs, and a Clustered Regularly Interspaced Short Palindromic Repeats-Associated (cas) protein, or nucleic acid sequence encoding a cas protein, wherein the one or more gRNAs uniquely hybridizes with the target HIV-1 DNA sequence, wherein the target HIV-1 DNA sequence is selected from the group consisting of SEQ ID NO: 1, SEQ ID NO: 2, and SEQ ID NO: 3; thereby inhibiting the function or presence of the target HIV-1 DNA sequence.

### BACKGROUND

The development of RNA-guided endonucleases (RGENs) including CRISPR/Cas9 for targeted excision of genomic DNA within eukaryotic cells has provided a potential approach for the permanent eradication of integrated viral pathogens. CRISPR/Cas9 gene disruption uses a guide RNA sequence that is complementary to an approximately 20 base pair target DNA sequence, together with the Cas endonuclease, to bind to and then cleave the target DNA region. For Streptococcus pyogenes Cas9 (SpCas9) to efficiently cleave double-stranded DNA, the target sequence to which the guide RNA binds must be located 5′ to the “N-G-G” nucleotide sequence that is termed the protospacer adjacent motif (PAM). Once cleaved by Cas9, endogenous cellular DNA repair mechanisms, most prominently non-homologous end joining (NHEJ), act on the double stranded breaks (DSBs), and through error prone repair mechanisms, can introduce small substitutions, insertions or deletions (indels). Large deletions or inversions can also be achieved through the introduction of two or more double-stranded breaks. In designing guide RNA sequences to viral pathogens, it is critically important to confirm that the guide RNA lacks complementarity to normal cellular genes, especially those that are important for cell growth, viability and metabolism. CRISPR-based therapeutics can target viral gene sequences that are either integrated into the host cell genome as in the case of HIV, or present in an extra-chromosomal body such as the covalently closed circular DNA (cccDNA) of the hepatitis B virus. Anti-viral CRISPR therapeutic strategies include manipulation of the host genome to improve immunity or resistance to viral infection, or the direct targeting of the integrated virus to excise some or all of the crucial components of the viral genome that would lead to interference of viral gene transcription. In the case of HIV-1, multiple groups have explored strategies to enhance HIV-1 resistance, most prominently by disrupting the genes that encode the chemokine co-receptors CCR5 or CXCR4, and by using approaches that inactivate or delete the HIV-1 provirus.

The predominant HIV-1 Major (M) group comprises multiple clades, which are genetic subtypes that vary in sequence within several areas of the HIV-1 genome including the long terminal repeat (LTR), as well as the env (envelope) and gag (group antigen) genes. There are currently 14 M group clades (A1, A2, A3, A4, A6, B, C, D, F1, F2, G, H, J, and K) and 97 reported circulating recombinant forms (CRFs) (hiv.lanl.gov/content/sequence/HIV/CRFs/CRFs.html). Individual subtypes can predominate within distinct geographic areas, making the design of virus-specific CRISPR-based therapies with universal clinical applicability challenging. For example, HIV-1_{A} is common in Eastern Africa, while HIV-1_{B} is the dominant form in Europe and the Americas. In Asia, HIV-1_{A} dominates in Russia, HIV-1_{C} is predominant in India, and numerous CRFs are found across the continent, especially in China. Thus, when considering the development of clinical therapeutic gene editing approaches, it is important to test guide RNAs against multiple clades within conserved regions of the genome.

### BRIEF SUMMARY

Disclosed are one or more of the guide RNAs described herein.

Disclosed are guide RNAs (gRNAs) that specifically bind a 5′ LTR human immunodeficiency virus-1 (HIV-1) sequence comprising TTGGATGGTGCTTCAAGTTA (SEQ ID NO: 1).

Disclosed are gRNAs that specifically bind a 5′ LTR HIV-1 sequence comprising

(SEQ ID NO: 2)
CTACAAGGGACTTTCCGCTG.

Disclosed are gRNAs that specifically bind a 5′ LTR HIV-1 sequence comprising

(SEQ ID NO: 3)
TCTACAAGGGACTTTCCGCT.

Disclosed are one or more of the nucleic acid sequences described herein.

Disclosed are nucleic acid sequences comprising a nucleic acid sequence encoding one or more gRNAs, wherein said one or more gRNAs hybridize with a target sequence in HIV-1, wherein the target sequence is selected from the group consisting of SEQ ID NO:1, SEQ ID NO:2, and SEQ ID NO:3.

Disclosed are one or more of the vectors described herein.

Disclosed are vectors comprising a nucleic acid sequence encoding one or more gRNAs, wherein the one or more gRNA hybridizes with a target sequence in HIV-1, wherein the target sequence is selected from the group consisting of SEQ ID NO:1, SEQ ID NO:2, and SEQ ID NO:3.

Disclosed are methods for inhibiting the function of a target HIV-1 DNA sequence in a cell comprising contacting a cell comprising a cellular genome and harboring a HIV-1 genome comprising a target HIV-1 DNA sequence integrated into the cellular genome with one or more gRNAs, or nucleic acids encoding said one or more gRNAs, and a Clustered Regularly Interspaced Short Palindromic Repeats-Associated (cas) protein, or nucleic acid sequence encoding a cas protein, wherein the one or more gRNAs uniquely hybridizes with the target HIV-1 DNA sequence, wherein the target HIV-1 DNA sequence is selected from the group consisting of SEQ ID NO:1, SEQ ID NO:2, and SEQ ID NO:3; thereby inhibiting the function or presence of the target HIV-1 DNA sequence.

Disclosed are methods for removing a target HIV-1 DNA sequence from a cellular genome comprising contacting a cell comprising a cellular genome and harboring a HIV-1 genome comprising a target HIV-1 DNA sequence integrated into the cellular genome with one or more gRNAs, or nucleic acids encoding said one or more gRNAs, and a Clustered Regularly Interspaced Short Palindromic Repeats-Associated (cas) protein, or nucleic acid sequence encoding a cas protein, wherein the one or more gRNAs uniquely hybridizes with the target HIV-1 DNA sequence, wherein the target HIV-1 DNA sequence is selected from the group consisting of SEQ ID NO:1, SEQ ID NO:2, and SEQ ID NO:3; thereby removing the target HIV-1 DNA sequence from the cellular genome.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example of the U3 region of 5′ LTR sequences. The sequence conservation of the four different target regions for the guide RNAs was derived by web alignments by hiv.lanl.gov utilizing 1242 complete HIV sequences from all clades. The logo for each target region indicates eh probability of each nucleotide at a specific position. The PAM sequence is boxed, and grey boxes denote sites of sequence insertions and deletions in multiple clades where alignment is lost, but becomes re-established downstream. The consensus sequence (shown in bold font) from 1,242 analyzed HIV-1 sequences was aligned with the sequence of NL4-3 (GenBank AF324493.2), the source of LTRs in the plasmid used in FIG. 2. The target region of the four guides are underlined, and the PAM sequence is in red font. The numbers in parentheses are coordinates with reference to the HIV-1 HBX2 sequence.

FIGS. 2A and 2B show maps of the two plasmids used in the in vitro cleavage assays. (FIG. 2A) Schematic representation of plasmid pNL-GFP with 5′ and 3′ LTRs (HIV-1 LTRs derived from NL4-3, shown as blue arrows) flanking the green fluorescent protein gene (eGFP in green). Noted are the restriction enzyme sites for Xmn I and Kpn I that cleave the plasmid into three fragments, with the anticipated fragment sizes indicated. Target sites of the LTR guides are noted by the numerical value that represents the nucleotide closest to the PAM. (FIG. 2B) Schematic representation of plasmid pBlue3′LTR-luc-B. This plasmid contains the LAI HIV-1 3′ LTR derived from pBluescript KS(+), shown as a blue arrow. This plasmid was used as the backbone for each clade, interchanging the 3′ LTR to correspond appropriately. Noted are the restriction enzyme sites for Xmn I and EcoRI that cleave the plasmid into two fragments, and the target sites of the LTR gRNA 127 and gRNA 363.

FIGS. 3A and 3B, shown an in vitro analysis of fragmented pNL-GFP cleaved with single or double LTR guide RNAs. Left panels: Gel images of fragmented pNL-GFP cleaved by (FIG. A) single or (FIG. B) double LTR guides directed to the 5′ and 3′ LTRs. Right panels: Cleavage efficiency of each guide tested was determined for the 5′ and 3′ LTR independently. Data are the mean±SEM from three experiments and analyzed using an unpaired two-tail T-test; *p≤0.05, **p≤0.01, ***p≤0.001, ****p≤0.0001. Results show cleavage of pNL-GFP cleaved by one LTR guide (right panel, FIG. 3A), and two LTR guides (right panel, FIG. 3B) at the 5′ and 3′ LTRs.

FIGS. 4A, 4B, 4C, and 4D show cleavage by individual gRNAs of the 3′ LTR from multiple HIV-1 clades. (A) Gel images of gRNA 127 (FIG. 4A, top panel) and gRNA 363 (FIG. 4A, bottom panel) cleavage of the 3′ LTR from pBlue 3′LTR-luc-A through G. (FIG. 4B) Cleavage efficiency of each clade against gRNA 127 (FIG. 4B, top panel) or gRNA 363 (FIG. 4B, bottom panel) was quantified. Data are the mean±SEM from three experiments and statistical analysis was determined using a one-way ANOVA with Tukey's HSD post-hoc test. All means were compared to one another. p-values are found in the table for clades cleaved with guide 363; *p≤0.05, **p≤0.01, ***p≤0.001, ****p≤0.0001. No significant differences were found between clades cleaved with gRNA 127. (FIG. 4C) DNA sequence differences (shown in red) between target sequence of gRNA 127 (top) and gRNA 363 (bottom) against the HIV-1 reference sequence, HXB2 (Target sequence). (FIG. 4D) Cutting Frequency Determination (CFD) was calculated using Python version 2.7 with packages pickle, re, and numpy. Original code was obtained from Doench, et al.

FIGS. 5A, 5B, and 5C show CRISPR/Cas9 cleavage of 5′LTR in TZM-bl cells with one or more LTR guide RNAs. Gene modification was analyzed using T7EI assay. (FIG. 5A) Example gel image yielded by T7E1 Assay which showed positive gene modification to the amplified 5′LTR and HPRT gene by guide RNAs 363 and 127, and in combination. (FIG. 5B) The expected fragment size of PCR product after T7E1 digestion. (FIG. 5C) Percent gene modification for each guide RNA treatment was determined using formula: 100×((1−(1−fraction cleaved))½). Mean data±SEM from six experiments. (FIG. 5D) Luciferase Reporter Assay mean data±SEM from triplicates. Statistical analysis was determined for both panels C and D using one-way ANOVA with Tukey's HSD post-hoc test. All means were compared to one another; *p≤0.05, **p≤0.01, ***p≤0.001, ****p≤0.0001.

FIGS. 6A, 6B, and 6C show an assessment of On and Off-Target Cleavage events using CIRCLE-Seq. CIRCLE-Seq was performed using DNA from TZM-bl cells and RNPs formed with recombinant SpCas9 and in vitro transcribed sgRNA 363 (FIG. 6A), and sgRNA 127 (FIG. 6B). Analysis of the resulting sequencing data identified the indicated cleavage events with the indicated numbers of read counts. (FIG. 6C) Annotation of cleavage events identified by CIRCLE-Seq. No off-target events were identified within the exons of protein coding genes. Off-target cleavage events occurred within both intergenic and intronic regions as indicated.

FIGS. 7A and 7B show an alignment of LTR sequences. (FIG. 7A) HXB2 5′ LTR (reference sequence) and pNL-GFP 5′LTR sequence alignment. (FIG. 7B) HXB2 5′LTR (reference sequence) and pNL-GFP 3′LTR sequence alignment. Guide target region and PAM region are denoted by blue and grey boxes, respectively. Mismatches are denoted by red font. Alignments were created by Serial Cloner 2.6.1.

FIGS. 8A and 8B show determining the cleavage efficiency of LTR guide 127 against the HIV-1 3′LTR of pNL-GFP. (FIG. 8A) Schematic representation of plasmid pNL-GFP with 5′ and 3′ LTRs (HIV-1 LTRs derived from NL4-3, shown as blue arrows) flanking the green fluorescent protein gene (GFP). Indicated are the restriction enzymes (Xmn I and Xho I) that cleave the plasmid, with the anticipated fragment sizes indicated and the target sites of the guides. (FIG. 8B) Agarose gel showing fragmented pNL-GFP being cleaved by LTR gRNA 127 at the 5′ and 3′ LTR. Cleavage efficiency of guide 127 was determined for the 3′ LTR. Cleavage efficiency data is shown in FIG. 3A.

FIGS. 9A and 9B show an output created by ICE software from DNA of TZM-bl cells cleaved with both gRNA 363 and gRNA 127. The 5′LTR of the modified HIV-1 was amplified using PCR as stated in methods for T7E1 Assay. PCR products were cleaned up using Monarch Nucleic Acid Purification kit (NEB, Ipswich, MA) and sequenced using the reverse primer (ACAGGCCAGGATTAACTGCG) on a Studio Seq Genetic Analyzer (ThermoFisher Scientific, Waltham, Ma). Samples were prepared for sequencing with BigDye™ Terminator v3. 1 Cycle Sequencing, and cleaned using BigDye XTerminator™ Purification Kit (ThermoFisher Scientific, Waltham, Ma) following the manufacturer's protocol. Ab files were imputed into ICE software for analysis (Synthego, Redwood City, CA). (FIG. 9A) Trace file segments of untreated (control) and targeted (gene edited) samples spanning the cut site of gRNA 363 and gRNA 127. The guide sequence is underlined and the PAM sequence is denoted by a red dotted line. The vertical dashed line denotes the cut site of each RNP. (FIG. 9B) Indels calculated by ICE and its relative prevalence within the sample targeted with gRNAs 363 and 127.

FIG. 10 shows nucleotide mismatches between the guide RNA and the target DNA region as it relates to in vitro cleavage efficiency. In vitro cleavage efficiencies using single gRNAs to either the 5′LTR (5′) or 3′LTR (3′) were plotted against the corresponding number (no.) of mismatches between guide RNA and target DNA sequences. As expected, the cleavage efficiencies were reduced as the number of mismatches increased.

FIG. 11 shows the conservation of guide RNA target region across HIV-1 clades. The target regions from clades A, B, C, D, F and G derived by filtered web alignments (http://www.hiv.lanl.gov) were aligned with gRNA 127, gRNA 363, gRNA361, and gRNA278. The percent occurrence of the desired nucleotide at each position for each guide is reported. If all isolates within a clade contained missing nucleotide information for a particular position, the term “gap” is noted. The PAM region is the first three nucleotides in gRNA 127 and gRNA278. The PAM region is the last three nucleotides in gRNA 363 and gRNA361.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a nucleic acid sequence” includes a plurality of such nucleic acid sequences, reference to “the guide RNA” is a reference to one or more guide RNAs and equivalents thereof known to those skilled in the art, and so forth.

The terms “polynucleotide” and “nucleic acid sequence,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

As used herein, the term “guide RNA”, “gRNA” “and “guide” are used interchangeably. In one embodiment, the gRNA can also be provided in the form of DNA encoding the gRNA.

As used herein, “Cas proteins” can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins.

As used herein, “selectively binds” is meant that a guide RNA or composition recognizes and physically interacts with its target (for example, LTR of HIV-1) and does not significantly recognize and interact with other targets. In some aspects, “specifically binds” as used throughout, can be used interchangeable with “selectively binds” or “specifically targets.”

By “treat” is meant to administer a nucleic acid sequence, vector, or composition of the invention to a subject, such as a human or other mammal (for example, an animal model), that has an increased susceptibility for being infected with HIV or developing AIDS, or that has an HIV infection or has AIDS, in order to prevent or delay a worsening of the effects of the disease, or to partially or fully reverse the effects of the disease.

By “prevent” is meant to minimize the chance that a subject who has an increased susceptibility for being infected with HIV or developing AIDS.

As used herein, the terms “administering” and “administration” refer to any method of providing a disclosed polypeptide, nucleic acid sequence, vector, composition, or a pharmaceutical preparation to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed conjugate so as to treat a subject or induce apoptosis. In an aspect, the skilled person can also alter or modify an aspect of an administering step so as to improve efficacy of a disclosed polypeptide, nucleic acid sequence, vector, composition, or a pharmaceutical preparation.

By an “effective amount” of a nucleic acid sequence, vector, or composition as provided herein is meant a sufficient amount of the nucleic acid sequence, vector, or composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of ordinary skill in the art using only routine experimentation. The term “therapeutically effective amount” means an amount of a therapeutic, prophylactic, and/or diagnostic agent that is sufficient, when administered to a subject suffering from or susceptible to a disease, disorder, and/or condition (e.g. AIDS), to treat, alleviate, ameliorate, relieve, alleviate symptoms of, prevent, delay onset of, inhibit progression of, reduce severity of, and/or reduce incidence of the AIDS disease, disorder, and/or condition.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

By “hybridizable” or “hybridize” or “complementary” or “substantially complementary” it is meant that a nucleic acid (e.g. RNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA]. In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Nucleic Acid Sequences

Disclosed herein, are guide RNA (gRNA) sequences. The disclosed gRNA sequences can be specific for one or more desired target sequences. In some aspects, the gRNA sequences can be specific to a target sequence, wherein the target sequence is a HIV-1 sequence. In some aspects the HIV-1 sequence can be a LTR sequence of HIV-1. For example, the target sequence can be one or more of SEQ ID NOs:1, 2, or 3. In some aspects, the gRNA sequence hybridizes with a target sequence in the genome of a cell. In some aspects, the cell can be a mammalian cell.

A guide sequence or single guide sequence (e.g. gRNA or sgRNA) can be any polynucleotide sequence having sufficient complementarity with a target sequence (polynucleotide sequence) to hybridize with the target sequence and direct sequence-specific binding of a CRISPR-Cas system or CRISPR complex to the target sequence. In some aspects, the degree of complementarity between a guide sequence (e.g. gRNA) and its corresponding target sequence is about or more than about 50%, 60%, 70%, 75%, 80%, 85%, 90%, 95%, 99%, or more. In some aspects, a guide sequence is about more than about 5, 10, 15, 20, 25, 30, 35, 40, 45, 50 or more nucleotides in length or any number in between. gRNA and sgRNA can be used interchangeably.

As used herein, the term “target sequence” refers to a sequence to which a guide sequence (e.g. gRNA/sgRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence can comprise any polynucleotide, such as DNA or RNA polynucleotides. In some aspects, a target sequence can be located in the nucleus or cytoplasm of a cell. In some aspects, the target sequence can be within an organelle of a eukaryotic cell (e.g., mitochondrion). A sequence or template that can be used for recombination into the targeted locus comprising the target sequences is referred to as an “editing template” or “editing polynucleotide” or “editing sequence.” In an aspect, the target sequence(s) can be selected from one or more of the nucleic acid sequences encoding a gene in a cell proliferation pathway. In an aspect, the target sequence(s) can be any sequence in which inhibition or modulation of the activity associated with the sequence would be beneficial for a subject. For example, as described herein, a target sequence can be a HIV-1 sequence, specifically a LTR sequence of HIV-1. In some aspects, the term “target sequence” and “gene of interest” can be used interchangeably. In some aspects, a target sequence is a target HIV-1 DNA sequence wherein inhibition or modulation of this sequence results in inhibiting the function or presence of HIV-1 in a cell.

Disclosed are gRNAs that specifically bind a 5′ LTR human immunodeficiency virus-1 (HIV-1) sequence comprising TTGGATGGTGCTTCAAGTTA (SEQ ID NO:1).

Disclosed are gRNAs that specifically bind a 5′ LTR HIV-1 sequence comprising

(SEQ ID NO: 2)
CTACAAGGGACTTTCCGCTG.

Disclosed are gRNAs that specifically bind a 5′ LTR HIV-1 sequence comprising

(SEQ ID NO: 3)
TCTACAAGGGACTTTCCGCT.

Disclosed are one or more of the nucleic acid sequences described herein.

Disclosed are nucleic acid sequences comprising a nucleic acid sequence encoding one or more gRNAs, wherein said one or more gRNAs hybridize with a target sequence in HIV-1, wherein the target sequence is selected from the group consisting of SEQ ID NO:1, SEQ ID NO:2, and SEQ ID NO:3.

In some aspects, disclosed are nucleic acid sequences comprising a nucleic acid sequence encoding one or more gRNAs, wherein said one or more gRNAs hybridizes with a target sequence in HIV-1, wherein the target sequence is selected from the group consisting of SEQ ID NO:1, SEQ ID NO:2, and SEQ ID NO:3.

Disclosed herein are gRNAs, wherein the gRNA hybridizes with a target sequence in HIV-1, wherein the target sequence is selected from the group consisting of SEQ ID NO:1, SEQ ID NO:2, and SEQ ID NO:3.

Disclosed herein are gRNAs that hybridize with a 5′ LTR human immunodeficiency virus-1 (HIV-1) sequence comprising TTGGATGGTGCTTCAAGTTA (SEQ ID NO:1).

Disclosed herein are gRNAs that hybridize with a 5′ LTR HIV-1 sequence comprising CTACAAGGGACTTTCCGCTG (SEQ ID NO:2).

Disclosed herein are gRNAs that hybridize with a 5′ LTR HIV-1 sequence comprising TCTACAAGGGACTTTCCGCT (SEQ ID NO:3).

Disclosed are target sequences comprising the sequence of SEQ ID NO:1, 2, or 3. The target sequence of a CRISPR complex can be any polynucleotide sequence endogenous or exogenous to the eukaryotic cell. For example, the target polynucleotide can be a polynucleotide residing in the nucleus of the eukaryotic cell. The target sequence can be a sequence coding a gene product (e.g., a protein) or a non-coding sequence (e.g., a regulatory polynucleotide or a junk DNA). In some aspects, the target sequence can be a sequence from a virus, such as HIV-1, that has infected a cell. It is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). A skilled person will be able to identify further PAM sequences for use with a given CRISPR enzyme. In an aspect, the PAM comprises NGG (where N is any nucleotide, (G)uanine, (G)uanine).

In some aspects, the gRNAs disclosed herein can further comprise a nucleic acid sequence that binds a Cas protein.

In some aspects, disclosed are nucleic acid sequences comprising one or more of the gRNA sequences disclosed herein and a sequence that encodes a Cas protein. In some aspects, a gRNA is a nucleic acid molecule that binds to a Cas endonuclease, forming a ribonucleoprotein complex (RNP), and targets the complex to a specific location within a target nucleic acid (e.g., a target sequence). It is to be understood that in some cases, a hybrid DNA/RNA can be made such that a gRNA includes DNA bases in addition to RNA bases, but the term “gRNA” is still used to encompass such a molecule herein.

As described herein, a gRNA can include two segments, a targeting segment (CRISPR RNA (crRNA)) and a protein-binding segment (transactivating crRNA (tracrRNA)). The targeting segment of a gRNA includes a nucleotide sequence (a guide sequence) that is complementary to (and therefore hybridizes with) a specific sequence (a target sequence) within a target nucleic acid (e.g., a viral genome). The protein-binding segment (or “protein-binding sequence”) interacts with (binds to) a Cas12d or Cas12e endonuclease. The protein-binding segment of a gRNA includes two complementary stretches of nucleotides that hybridize to one another to form a double stranded RNA duplex (dsRNA duplex), or stem loop. Site-specific binding and/or cleavage of a target nucleic acid (e.g., viral DNA) can occur at locations (e.g., target sequence of a target locus) determined by base-pairing complementarity between the gRNA (the guide sequence of the gRNA) and the target sequence of the target nucleic acid.

A gRNA and a Cas endonuclease form a complex (e.g., bind via non-covalent interactions). The gRNA provides target specificity to the complex by including a targeting segment, which includes a guide sequence (a nucleotide sequence that is complementary to a target sequence of a target nucleic acid). The Cas endonuclease of the complex provides the site-specific activity (e.g., cleavage activity provided by the Cas endonuclease). In other words, the Cas endonuclease is guided to a target nucleic acid sequence (e.g. a target sequence) by virtue of its association with the gRNA.

In some aspects, a gRNA can be a single guide RNA (sgRNA) that comprises both the crRNA and the tracrRNA. In some aspects, a gRNA can be formed after a crRNA and a tracrRNA hybridize (e.g. they have complementary segments) thus allowing the targeting sequence of the crRNA to bind to the target sequence while the protein binding segment of the tracrRNA brings the endonuclease which can then cleave the target sequence.

The targeting segment of a gRNA includes a guide sequence (i.e., a targeting sequence), which is a nucleotide sequence that is complementary to a sequence (a target sequence e.g. SEQ ID NO:1, SEQ ID NO:2, or SEQ ID NO:3) in a target nucleic acid. In other words, the targeting segment of a gRNA can interact with a target nucleic acid (e.g., viral genome) in a sequence-specific manner via hybridization (i.e., base pairing). The guide sequence of a gRNA can be modified (e.g., by genetic engineering)/designed to hybridize to any desired target sequence (e.g., while taking the PAM into account, e.g., when targeting a dsDNA target) within a target nucleic acid (e.g., viral genome).

In some embodiments, the percent complementarity between the guide sequence and the target sequence of the target nucleic acid is 60% or more (e.g., 65% or more, 70% or more, 75% or more, 80% or more, 85% or more, 90% or more, 95% or more, 97% or more, 98% or more, 99% or more, or 100%). In some cases, the percent complementarity between the guide sequence and the target sequence of the target nucleic acid is 80% or more (e.g., 85% or more, 90% or more, 95% or more, 97% or more, 98% or more, 99% or more, or 100%). In some cases, the percent complementarity between the guide sequence and the target sequence of the target nucleic acid is 90% or more (e.g., 95% or more, 97% or more, 98% or more, 99% or more, or 100%). In some cases, the percent complementarity between the guide sequence and the target sequence of the target nucleic acid is 100%.

In some cases, the percent complementarity between the guide sequence and the target sequence of the target nucleic acid is 60% or more (e.g., 70% or more, 75% or more, 80% or more, 85% or more, 90% or more, 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target sequence of the target nucleic acid is 80% or more (e.g., 85% or more, 90% or more, 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target sequence of the target nucleic acid is 90% or more (e.g., 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target sequence of the target nucleic acid is 100% over 19 or more (e.g., 20 or more, 21 or more, 22 or more) contiguous nucleotides.

In some cases, the percent complementarity between the guide sequence and the target sequence of the target nucleic acid is 60% or more (e.g., 70% or more, 75% or more, 80% or more, 85% or more, 90% or more, 95% or more, 97% or more, 98% or more, 99% or more, or 100%) over 19-25 contiguous nucleotides. In some cases, the percent complementarity between the guide sequence and the target sequence of the target nucleic acid is 80% or more (e.g., 85% or more, 90% or more, 95% or more, 97% or more, 98% or more, 99% or more, or 100%) over 19-25 contiguous nucleotides. In some cases, the percent complementarity between the guide sequence and the target sequence of the target nucleic acid is 90% or more (e.g., 95% or more, 97% or more, 98% or more, 99% or more, or 100%) over 19-25 contiguous nucleotides. In some cases, the percent complementarity between the guide sequence and the target sequence of the target nucleic acid is 100% over 19-25 contiguous nucleotides.

In some cases, the guide sequence has a length in a range of from 19-30 nucleotides (nt) (e.g., from 19-25, 19-22, 19-20, 20-30, 20-25, or 20-22 nt). In some cases, the guide sequence has a length in a range of from 19-25 nucleotides (nt) (e.g., from 19-22, 19-20, 20-25, 20-25, or 20-22 nt). In some cases, the guide sequence has a length of 19 or more nt (e.g., 20 or more, 21 or more, or 22 or more nt; 19 nt, 20 nt, 21 nt, 22 nt, 23 nt, 24 nt, 25 nt, etc.). In some cases the guide sequence has a length of 19 nt. In some cases the guide sequence has a length of 20 nt. In some cases the guide sequence has a length of 21 nt. In some cases the guide sequence has a length of 22 nt. In some cases the guide sequence has a length of 23 nt.

### C. Vectors

Disclosed are one or more of the vectors described herein. For example, disclosed are vectors comprising a nucleic acid sequence comprising a gRNA.

Disclosed are vectors comprising a nucleic acid sequence encoding one or more gRNAs, wherein the one or more gRNA hybridizes with a target sequence in HIV-1, wherein the target sequence is selected from the group consisting of SEQ ID NO:1, SEQ ID NO:2, and SEQ ID NO:3.

Disclosed are vectors comprising a nucleic acid sequence comprising a gRNA and further comprising at least one marker gene.

Disclosed are vectors comprising a nucleic acid sequence comprising a gRNA and a nucleic acid sequence encoding a Cas protein.

Disclosed are vectors comprising a nucleic acid sequence encoding a Cas protein.

In some aspects, the disclosed vectors are expression vectors. In some aspects, the expression vector can be a viral vector such as a Lentiviral vector. In some aspects, the vector can be any of those described herein.

The vectors disclosed herein can be viral or non-viral vectors or any type of expression vector. Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding a Cas protein. In some aspects, the vectors can also deliver a gRNA.

There are a number of compositions and methods which can be used to deliver nucleic acids, such as guide RNAs, to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding one or more of the disclosed mutated Cas9 proteins operably linked to a control element.

The “control elements” present in an expression vector are those non-translated regions of the vector-enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

Preferred promoters controlling transcription from vectors in mammalian host cells may be obtained from various sources, for example, the genomes of viruses such as polyoma, Simian Virus 40 (SV40), adenovirus, retroviruses, hepatitis B virus and most preferably cytomegalovirus, or from heterologous mammalian promoters (e.g., beta actin promoter). The early and late promoters of the SV40 virus are conveniently obtained as an SV40 restriction fragment, which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Additionally, promoters from the host cell or related species can also be used.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osbome, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, a-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product is used to determine if the gene has been delivered to the cell and once delivered is being expressed. Preferred marker genes are the E. coli lacZ gene, which encodes ß-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a guide RNA, into a cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

Other useful systems include, for example, replicating and host-restricted non-replicating vaccinia virus vectors. In addition, the disclosed nucleic acid sequences can be delivered to a target cell in a non-nucleic acid based system. For example, the disclosed nucleic acid sequences and constructs can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### D. Compositions

Disclosed are compositions comprising the target sequences, nucleic acid sequences (e.g. guide RNAs or sequences capable of encoding the guide RNA sequences) or vectors described herein. For example, disclosed are compositions comprising vectors, wherein the vectors comprise any of the nucleic acid sequences disclosed herein.

1. Pharmaceutical Compositions

In some aspects, the disclosed compositions further comprise a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

2. Delivery of Compositions

In the methods described herein, delivery (or administration) of the compositions to a subject or cells can be via a variety of mechanisms. As defined above, any one or more of the guide RNAs or vectors described herein can be used to produce a composition which can also include a carrier such as a pharmaceutically acceptable carrier. For example, disclosed are pharmaceutical compositions, comprising the guide RNAs disclosed herein, and a pharmaceutically acceptable carrier.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed nucleic acid constructs and vectors.

### E. Methods

Disclosed are methods for altering, modifying or inhibiting the function of a target HIV-1 DNA sequence in a cell.

Disclosed are methods for inhibiting the function of a target HIV-1 DNA sequence in a cell comprising contacting a cell comprising a cellular genome and harboring a HIV-1 genome comprising a target HIV-1 DNA sequence integrated into the cellular genome with one or more gRNAs, or nucleic acids encoding said one or more gRNAs, and a cas protein, or nucleic acid sequence encoding a cas protein, wherein the one or more gRNAs uniquely hybridizes with the target HIV-1 DNA sequence, wherein the target HIV-1 DNA sequence is any one or more of those described herein; thereby inhibiting the function or presence of the target HIV-1 DNA sequence.

Disclosed are methods for inhibiting the function of a target HIV-1 DNA sequence in a cell comprising contacting a cell comprising a cellular genome and harboring a HIV-1 genome comprising a target HIV-1 DNA sequence integrated into the cellular genome with one or more gRNAs, or nucleic acids encoding said one or more gRNAs, and a Clustered Regularly Interspaced Short Palindromic Repeats-Associated (cas) protein, or nucleic acid sequence encoding a cas protein, wherein the one or more gRNAs uniquely hybridizes with the target HIV-1 DNA sequence, wherein the target HIV-1 DNA sequence is selected from the group consisting of SEQ ID NO:1, SEQ ID NO:2, and SEQ ID NO:3; thereby inhibiting the function or presence of the target HIV-1 DNA sequence.

Disclosed are methods for removing a target HIV-1 DNA sequence from a cellular genome.

Disclosed are methods for removing a target HIV-1 DNA sequence from a cellular genome comprising contacting a cell comprising a cellular genome and harboring a HIV-1 genome comprising a target HIV-1 DNA sequence integrated into the cellular genome with one or more gRNAs, or nucleic acids encoding said one or more gRNAs, and a Clustered Regularly Interspaced Short Palindromic Repeats-Associated (cas) protein, or nucleic acid sequence encoding a cas protein, wherein the one or more gRNAs uniquely hybridizes with the target HIV-1 DNA sequence, wherein the target HIV-1 DNA sequence is any one or more of those described herein; thereby removing the target HIV-1 DNA sequence from the cellular genome.

Disclosed are methods for removing a target HIV-1 DNA sequence from a cellular genome comprising contacting a cell comprising a cellular genome and harboring a HIV-1 genome comprising a target HIV-1 DNA sequence integrated into the cellular genome with one or more gRNAs, or nucleic acids encoding said one or more gRNAs, and a Clustered Regularly Interspaced Short Palindromic Repeats-Associated (cas) protein, or nucleic acid sequence encoding a cas protein, wherein the one or more gRNAs uniquely hybridizes with the target HIV-1 DNA sequence, wherein the target HIV-1 DNA sequence is selected from the group consisting of SEQ ID NO:1, SEQ ID NO:2, and SEQ ID NO:3; thereby removing the target HIV-1 DNA sequence from the cellular genome.

Disclosed are methods for treating a subject infected with HIV-1. Disclosed are methods for treating a subject infected with HIV-1 comprising administering to a subject one or more gRNAs, or nucleic acids encoding said one or more gRNAs, and a cas protein, or nucleic acid sequence encoding a cas protein, wherein the subject has an HIV-1 DNA sequence integrated into the genome, wherein the one or more gRNAs uniquely hybridizes with the HIV-1 DNA sequence; thereby removing the HIV-1 DNA sequence from the genome.

Disclosed are methods for treating a subject infected with HIV-1. Disclosed are methods for treating a subject infected with HIV-1 comprising administering to a subject one or more gRNAs, or nucleic acids encoding said one or more gRNAs, and a cas protein, or nucleic acid sequence encoding a cas protein, wherein the subject has an HIV-1 DNA sequence integrated into the genome, wherein the one or more gRNAs uniquely hybridizes with the HIV-1 DNA sequence, wherein the target HIV-1 DNA sequence is selected from the group consisting of SEQ ID NO:1, SEQ ID NO:2, and SEQ ID NO:3; thereby removing the target HIV-1 DNA sequence from the cellular genome.

In some aspects, the one or more gRNAs do not bind or hybridize to the cellular genome. Thus, in some aspects, the gRNAs only bind or hybridize to a HIV-1 sequence, for example a HIV-1 LTR sequence.

In some aspects, the gRNAs disclosed herein can target a LTR region of two or more HIV clades. In some aspects, gRNAs disclosed herein hybridize to a target HIV-1 DNA sequence in the LTR region of two or more HIV clades. For example, the HIV clades can be two or more of any of the known clades. For example, in some aspects, HIV clades A to G can be targeted by the disclosed gRNAs. Thus, disclosed are methods of targeting two or more HIV-1 clades using the gRNAs and target sequences described herein.

In some aspects, the target HIV-1 DNA sequence is SEQ ID NO: 1, and wherein the one or more guide RNA, or nucleic acids encoding the one or more guide RNA comprise the sequence of SEQ ID NO:1, or the complement thereof.

In some aspects, the target HIV-1 DNA sequence is SEQ ID NO:2, and wherein the one or more guide RNA, or nucleic acids encoding the one or more guide RNA comprise the sequence of SEQ ID NO:2, or the complement thereof.

In some aspects, the target HIV-1 DNA sequence is SEQ ID NO:3, and wherein the one or more guide RNA, or nucleic acids encoding the one or more guide RNA comprise the sequence of SEQ ID NO:3, or the complement thereof.

In some aspects, the one or more guide RNA and the cas protein form a complex inside the cell, and wherein the complex cuts the HIV-1 DNA sequence, thereby inhibiting the function or presence of the target HIV-1 DNA sequence. In some aspects, the complex cuts the HIV-1 DNA sequence at the 5′LTR and the 3′LTR, thereby inhibiting the function or presence of the target HIV-1 DNA sequence. Because the 5′ and 3′ LTRs are repeats on either end of the HIV-1 genome, cutting the HIV-1 at the LTR can result in cleaving the majority of the HIV-1 genome from a host sequence.

In some aspects, the methods comprise administering a nucleic acid sequence encoding a cas protein, administering a cas protein or administering a vector that encodes a cas protein to a subject. In some aspects, the methods comprise contacting a cell with a nucleic acid sequence encoding a cas protein, a cas protein or a vector that encodes a cas protein. In some aspects, the cas protein can be cas9. In some aspects, any of the disclosed cas proteins can be used in the disclosed methods. In some aspects, the cas protein has been codon-optimized for expression in human cells. In some aspects, the cas protein further comprises a nuclear localization sequence.

In some aspects, the nucleic acids encoding the one or more guide RNA, and the nucleic acids encoding the cas protein are contained in an expression vector. In some aspects, the expression vector is a viral vector.

In some aspects, contacting comprises contacting a cell with one or more expression vectors comprising the nucleic acids encoding the one or more guide RNA and the nucleic acids encoding the cas protein. In some aspects, the contacting step is carried out in vitro. In some some aspects, the contacting step is carried out in vivo. Thus, upon contact with the expression vectors, in some aspects, the cells can be in culture or in a subject.

In some aspects, two gRNAs can be used in the disclosed methods. The first gRNA can be complementary to a first target sequence and a second gRNA can be complementary to a second target sequence in the viral genome. In some aspects, a single gRNA can be complementary to a first target sequence and a second target sequence in a viral genome when the viral genome has repeat sequences. For example, this can happen with retroviruses, such as HIV-1 described herein, having long terminal repeats (LTRs) at each end (5′ and 3′) of the viral genome wherein the LTR is the same at the 5′ end of the viral genome and the 3′ end of the viral genome. Therefore, a gRNA can be complementary to a single sequence that is present at both the 5′ end of the viral genome and 3′ end of viral genome. For example, a first target sequence and a second target sequence can be a single sequence within the LTR. A first target sequence can be present in the 5′ LTR while the second target sequence can be present in the 3′ LTR.

In some aspects, the Cas protein and gRNA are expressed from different vectors/constructs. Thus, in some aspects, at least two different constructs can be used. In some aspects, the Cas protein and gRNA are expressed from the same construct. For example, a construct can comprise a nucleic acid sequence, wherein the nucleic acid sequence comprises at least two elements, wherein a first element comprises a nucleic acid sequence that encodes Cas9 and a second element comprises a nucleic acid sequence that expresses a gRNA.

As used herein, “Cas proteins” can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins.

In some aspects, the Cas protein can be a Cas9 protein. In some aspects, the Cas9 can be a Streptococcus pyogenes Cas9 (SpCas9). The Streptococcus pyogenes Cas9. Examples of various Cas9 guide RNAs can be found in the art, and in some cases variations similar to those introduced into Cas9 guide RNAs can also be introduced into Cas12d or Cas12e gRNAs of the present disclosure. For example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

In some aspects, the disclosed methods use a CRISPR or CRISPR-Cas system. As used herein, “CRISPR system” and “CRISPR-Cas system” refers to transcripts and other elements involved in the expression of or directing the activity of CRISPR-associated (“Cas”) genes, including sequences encoding a Cas gene, a guide sequence (also referred to as a “spacer” in the context of an endogenous CRISPR system; e.g. guide RNA or gRNA), or other sequences and transcripts from a CRISPR locus. In some aspects, one or more elements of a CRISPR system is derived from a type I, type II, or type III CRISPR system. In some aspects, one or more elements of a CRISPR system are derived from a particular organism comprising an endogenous CRISPR system, such as Streptococcus pyogenes. Generally, a CRISPR system is characterized by elements that promote the formation of a CRISPR complex at the site of a target sequence (also referred to as a proto spacer in the context of an endogenous CRISPR system).

In some aspects, the gRNA targets and hybridizes with the target sequence and directs a RNA-directed nuclease to the DNA locus. In some aspects, the CRISPR-Cas system and vectors disclosed herein comprise one or more gRNA sequences. In some aspects, the CRISPR-Cas system and vectors disclosed herein comprise 2, 3, 4 or more gRNA sequences. In some aspects, the CRISPR-Cas system and/or vector described herein comprises 4 gRNA sequences in a single system. In some aspects, the gRNA sequences disclosed herein can be used to modulate HIV-1 infection or replication.

The compositions described herein can include a nucleic acid encoding a RNA-directed nuclease. The RNA-directed nuclease can be a CRISPR-associated endonuclease. In some aspects, the RNA-directed nuclease is a Cas9 nuclease or protein. In some aspects, the Cas9 nuclease or protein can have a sequence identical to the wild-type Streptococcus pyrogenes sequence. In some aspects, the Cas9 nuclease or protein can be a sequence for other species including, for example, other Streptococcus species, such as thermophilus; Pseudomonas aeruginosa, Escherichia coli, or other sequenced bacteria genomes and archaea, or other prokaryotic microogranisms. In some aspects, the wild-type Streptococcus pyrogenes sequence can be modified. In some aspects, the nucleic acid sequence can be codon optimized for efficient expression in eukaryotic cells.

Disclosed herein, are CRISPR-Cas systems, referred to as CRISPRi (CRISPR interference), that utilizes a nuclease-dead version of Cas9 (dCas9). In some aspects, the dCas9 can be used to repress expression of one or more target sequences (e.g., tumor necrosis factor receptor (e.g., TNFR2), interleukin 1 receptor (e.g, IL1R2, IL6R), A-kinase anchor protein 5 (e.g., AKAP5, a glycoprotein (e.g., gp130) and transient receptor potential cation channel subfamily V member 1 (TRPV1)). Instead of inducing cleavage, dCas9 remains bound tightly to the DNA sequence, and when targeted inside an actively transcribed gene, inhibition of, for example, pol II progression through a steric hindrance mechanism can lead to efficient transcriptional repression. In some aspects, the dCas9 can be used to induce expression of one or more target sequences (e.g., PTEN, MYC).

In some aspects, the CRISPR system can be used in which the nucleus has been deactivated. Further, a KRAB, VPR or p300 core can be attached. In some aspects, the KRAB is attached to downregulate one or more genes in a cell. In some aspects, the p300core or VPR can be attached to upregulate one or more genes in a cell.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for producing vectors comprising the disclosed nucleic acid sequences.

The disclosed kits can also include one or more of the disclosed nucleic acid sequences (e.g. guide RNAs).

Disclosed are kits, comprising: one or more guide RNA, or nucleic acids encoding the one or more guide RNA, wherein the guide RNA hybridizes with a target HIV-1 DNA sequence; and a cas protein, or a nucleic acid encoding the cas protein. In some aspects, the guide RNA or cas protein can be any of those disclosed herein.

### Examples

1. Introduction

Herein the in vitro and in vivo cleavage efficacy of a panel of SpCas9 guide RNAs targeting the proviral LTR region were compared, and the broad applicability of this panel of guides to cleave the LTR region from disparate clades of HIV-1 assessed. To define both on- and off-target cleavage efficiency, genomic DNA containing integrated HIV-1 provirus was subjected to CIRCLE-Seq analysis as a method to quantify and identify specific and off-target genomic cleavage events. CIRCLE-Seq is a highly sensitive approach that uses bioinformatics to quantify DNA cleavage following gene editing by CRISPR/Cas. A high degree of gene cleavage with several single guide RNAs, which in some cases, was increased when two guides were used together. Moreover, a particular guide designed was able to cleave the 5′ HIV-1 LTR region from multiple HIV-1 clades. CIRCLE-Seq analyses revealed very few predicted off-target events. These findings underscore the importance of testing guide RNAs against genetically disparate targets to identify broadly conserved regions among genetically disparate HIV-1 sources, and to confirm lack of off-target events in non-targeted regions. Finally, an anti-HIV-1 guide nomenclature was proposed to standardize the naming of guide RNAs among research laboratories in which gRNAs are named based on location of the target DNA region.

2. Materials and Methods

i. Identification of Guide RNA Sequences to the HIV-1 Provirus.

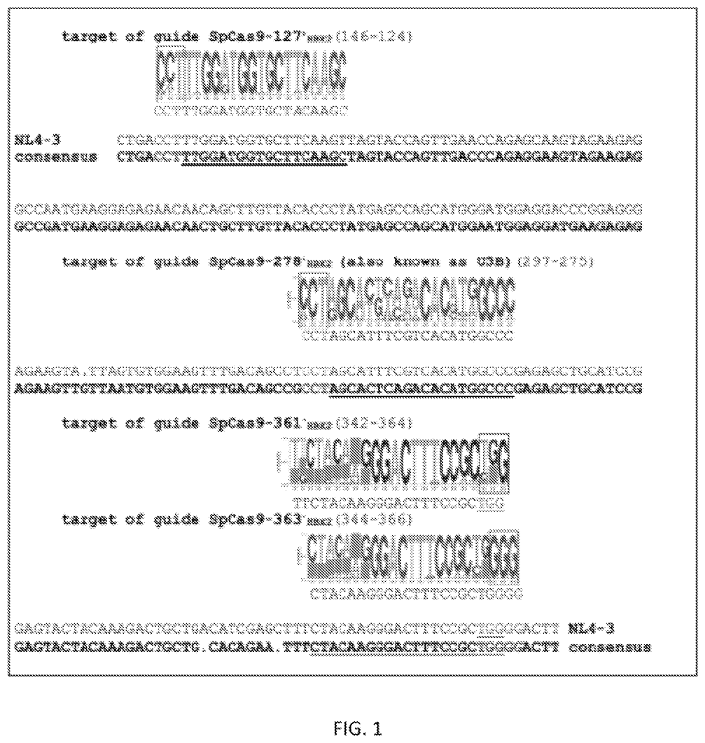

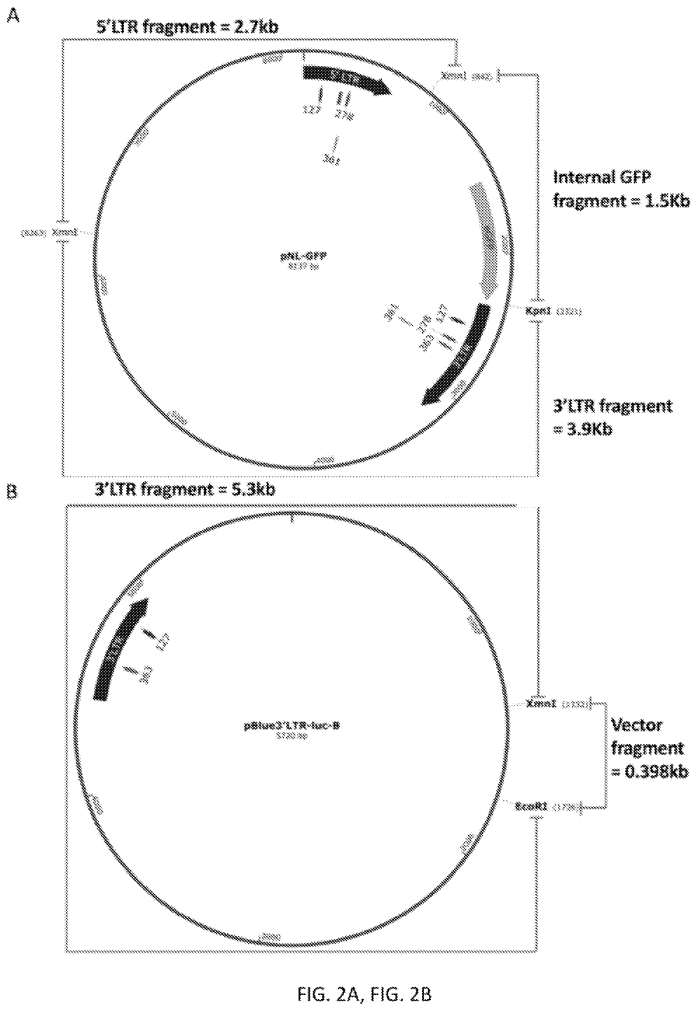

To develop gRNA candidates for HIV-1 excision, in silico approaches were used to identify regions that could serve as SpCas9 targets. Two methods were used to identify candidate guide RNA (gRNA) target sequences. The first method searched for possible target regions in the pNL4-3 HIV 5′ LTR by scanning for the Cas9 PAM (NGG) using Gene Construction Kit software (Textco Biosoftware, Raleigh, NC), and then testing each of the adjacent 20 base pair (bp) sequences for unintended homologies to human genomic DNA using Blast from the National Center for Biotechnology Information's website. Using this process, the U3B gRNA (SpCas9-278⁺_{HXB2}) was identified, and subsequently cloned by the Gibson method (New England Biolabs, Ipswich, MA, catalog #E2611S) into a vector containing the guide RNA scaffold under the human U6 promoter (gRNA Cloning Vector, Addgene plasmid #41824; RRID: Addgene 41824)). The second method identified gRNA target sequences using Integrated DNA Technologies (IDT) custom gRNA design link. The sequence for the HXB2 5′ LTR region was uploaded into the IDT site, and this method identified the gRNAs noted as SpCas9-127⁺_{HXB2}, SpCas9-361-HXB2, and SpCas9-363-HXB2. The target regions for these gRNAs are shown in FIG. 1 and FIG. 2. Cas-OFFinder was used to determine the possible number of off-target events (Table 1). Conservation of these target regions between HIV-1 isolates were determined using web alignments from the Los Alamos National Laboratory HIV Sequence Database (hiv.lanl.gov/content/index). All clades were represented in the 1,242 HIV sequences used (data that was available in 2017). A logo graphical representation of the probability of each nucleotide at its specific base pair position was created (FIG. 1).

TABLE 1
Number of off-target events within the human genome
determined by Cas-OFFinder based on the number of
mismatches between target region and guide RNA
No. of mismatches (M) in target region to guide

| Guide | | | | | | |
|---|---|---|---|---|---|---|
| RNA name | 1M | 2M | 3M | 4M | 5M | 6M |
| SpCas9- | 0 | 0 | 6 | 52 | 597 | 5318 |
| 127 + HXB2 | | | | | | |
| SpCas9- | 0 | 1 | 5 | 65 | 687 | 5590 |
| 287 + HXB2 | | | | | | |
| SpCas9- | 0 | 1 | 38 | 51 | 528 | 4075 |
| 361 − HXB2 | | | | | | |
| SpCas9- | 0 | 0 | 6 | 61 | 784 | 9257 |
| 363 − HXB2 | | | | | | |

Standardization of gRNA nomenclature. Guide RNA nomenclature was developed for the SpCas9 gRNAs utilized in this study. The species origin of the Cas enzyme is noted first, followed by the nucleotide position adjacent to the Cas-specific PAM. The orientation of the complementary strand the guide RNA binds to is denoted in superscript as being either on the plus(+) strand (5′→3′) or the minus (−) strand (3′→5′). In the case of the gRNAs reported here, the numerical designation of the gRNA refers to the nucleotide position in the HBX2-HIV reference genome (accession no. K03455.1). The reference genome used is depicted as a subscript notation.

ii. In Vitro DNA Cleavage Assay

Several guide RNAs were designed with specificity to the HIV-1 LTR (FIG. 1 shows the target sequences and FIG. 2 shows the sites in pNL-GFP), and tested cleavage efficiency either with a single gRNA or with a combination of two gRNAs. The target plasmid, pNL-GFP (FIG. 2), was derived from pNL4-3 Luc (Addgene #3418) by digesting with NsiI and XhoI to remove the majority of the HIV and luciferase sequences. Nuclear localization and splice acceptor sites were then reintroduced using PCR, and added the eGFP sequence by PCR from the Clontech plasmid (pEGFP-N1) between the XhoI and KpnI sites. In addition, we tested two different gRNAs against pBluescript KS(+)LTR-luc plasmids expressing various HIV-1 3′ LTRs from HIV-1 clades A through G (NIH AIDS repository, catalog numbers 4787-4793).

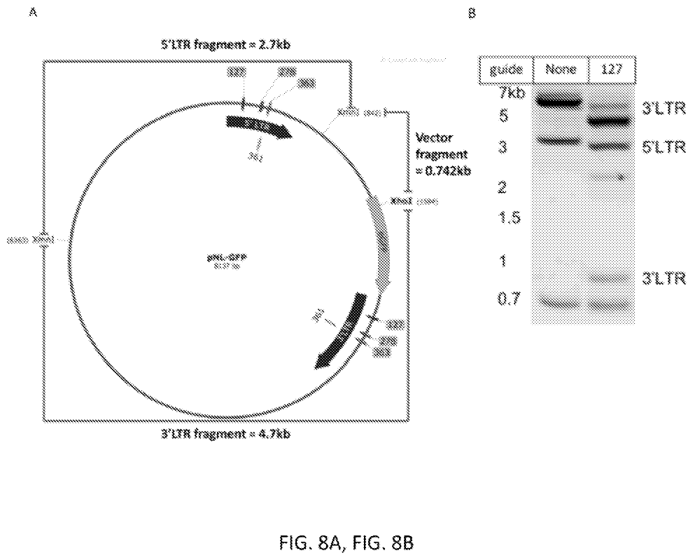

To test HIV-1 DNA cleavage, the pNL-GFP plasmid was first digested either with combinations of the Kpn1 and Xmn1 restriction enzymes, or the Xho1 and Xmn1 restriction enzymes (NEB, Ipswich, MA), to yield three fragments (FIG. 2 and FIG. 8). The pBluescript KS(+) plasmids expressing different HIV-1 clades (A-G) were each digested with the combination of the Xmn1 and EcoRI restriction enzymes to generate two fragments of 6 kB and 0.4 kB. The in vitro cleavage assay was performed by initially forming a duplex of gRNA and tracrRNA, followed by the addition of recombinant SpCas9 to form a ribonucleoprotein complex (RNP). Briefly, the gRNA:tracrRNA duplex was formed by incubating 300 pmoles of gRNA with 300 pmoles of tracrRNA (Alt-R® CRISPR-Cas9 tracrRNA, IDTdna Coralville, IA) for 5 min at 95° C. This duplex was then diluted 1:100 with nuclease free duplex buffer (IDT DNA) to a final concentration of 3 μM. For a 30 μl RNP assembly, 1 μl (3 pmoles) of gRNA:tracrRNA duplex was added to 3 pmoles of Cas9 (Alt-R® S.p. Cas9 Nuclease V3, IDTdna) together with 3 μl of 10×Cas9 nuclease reaction buffer (IDT). The complexes were incubated at room temperature for 15 min, and then added to 300 ng of the digested plasmid DNA and incubated at 37° C. for an additional 15 min. The reaction was stopped by the addition of 1 μl of proteinase K (800 units/ml, NEB) and incubated at room temperature for 10 min. To assess cleavage efficiency, the DNA fragments were separated on a 0.9% agarose gel, and visualized with GelRed (Biotium, Fremont, CA) on a Biorad Versa Doc imaging system using Image Lab software (Hercules, CA).

iii. In Vivo Cleavage of the HIV-1 LTR

TZM-bl cells were used as a model for the in vivo assessment of CRISPR/Cas9 gene cleavage as these cells contain two copies of a modified HIV-1 provirus that express either the luciferase or beta-galactosidase gene. These cells were maintained in DMEM supplemented with 10% fetal bovine serum (FBS), 2 mM glutamine, and 1× penicillin-streptomycin (GIBCO, Grand Island, NY) at 37° C. and 5% CO2. A mixture of RNPs containing either one or two different gRNAs to the U3 region of the LTR (gRNA 363 and gRNA127) were transfected into TZM-bl cells using CRISPRmax (CMAX0001, ThermoFisher, Waltham, MA). Control RNPs were prepared using a gRNA to HPRT (hypoxanthine phosphoribosyltransferase) (IDT, Coralville, IA). Briefly, RNPs for TZM-bl cells were prepared by mixing 40 pmoles of the gRNA:tracrRNA duplex, 40 pmoles of Cas9, and 3.4 μl of Cas9-plus reagent (total volume equals 83 μl), and incubating for 5 minutes at room temperature. This RNP was then mixed with 4 μl of CRISPRMax plus 79 μl of OPTI Mem (Gibco), and incubated for an additional 20 minutes. The RNP was then added to wells of a 24 well plate, followed by the addition of 8×104 TZM-bl cells in DMEM supplemented with 1% FBS in a final volume of 0.5 ml. Cells were incubated for 60 hours prior to analysis of gene cleavage.

The loss of functional activity in TZM-bl cells following in vivo cleavage of the HIV-1 LTR was assessed by a luciferase reporter assay. Briefly, TZM-bl cells transfected with RNPs (above) were removed using trypsin following the 60 hr incubation, counted, and 1×104 cells from each transfection condition plated in triplicate in wells of a 96 well plate. The cells were allowed to attach to the plastic wells overnight, and then stimulated with 10 ng/ml of TNF-α for 4 hours, washed with 1× phosphate buffered saline (PBS), and lysed with 25 μl of luciferase cell culture lysis reagent (Promega). The plate containing the cells was placed at −80° C. overnight to facilitate lysis, then thawed at room temperature in the dark. Twenty μl of each lysate was then transferred to 1.5 ml microliter tubes, followed by the addition of 100 μl of Luciferase assay substrate (Promega). The luciferase activity was recorded in a luminometer (Turner Systems 20/20).

iv. T7E1 Assay

DNA was isolated from TZM-bl cells using the QIAmp Micro DNA kit (Qiagen, Waltham, MA) following in vivo transfection of RNPs. Genome editing via the CRISPR/Cas9 RNP complex was quantified by the EnGen Mutation detection kit (NEB) according to the manufacturer's instructions. Briefly, PCR was performed on genomic DNA flanking the 5′ LTR target site for 35 cycles (98° C., 30 sec; 66° C., 20 sec; 72° C., 30 sec) using Phusion Hi-Fidelity DNA polymerase (NEB), primer pairs (fwd: GGAAGGGCTAATTCACTCCCAA, rev: ACAGGCCAGGATTAACTGCG) at a final concentration of 500 nM, and 50-100 ng of genomic DNA. A 1.083 kb portion of the HPRT gene was amplified using Q5 Hot Start High Fidelity 2× Master Mix (NEB), and Alt-R® Human HPRT PCR Primer Mix (IDT). This PCR was performed for 35 cycles (98° C., 15 sec; 67° C., 20 sec; 72° C., 30 sec). To complete the assay, PCR products were re-annealed (95°−85° C., 2° C./sec; 85° C.-25° C., 0.1° C./sec) in a final volume of 19 μl using 5 μl of PCR product, 2 μl of 10×NEB Buffer 2, and then digested with 1 μl of EnGen T7E1 for 15 mins at 37° C. The reaction was stopped by incubating with 1 μl of proteinase K (NEB) for 5 mins at 37° C., and PCR products were resolved on a 1.5% agarose gel. DNA bands were visualized with GelRed, and band density was determined as described above. The quantification of gene modification was based on relative band intensity and determined by the formula: % Gene Modification=100×(1−fraction cleaved)^{1/2}.

v. CIRCLE-Seq

Single guide (sg) RNA Synthesis. The guide RNAs used for CIRCLE-Seq in vitro cleavage reactions were single guide RNAs (sgRNA) containing both the target-specific gRNA and the tracrRNA. These were transcribed from a dsDNA template with a T7 promoter using the Engen sgRNA synthesis kit (NEB, catalog #E3322S) and purified using the Monarch RNA Cleanup kit (NEB, catalog #T2040L). DNA oligos containing the T7 promoter and target-specific sequence required for synthesis of the dsDNA template were purchased from ThermoFisher (catalog #10336022).

vi. CIRCLE-Seq Library Preparation

Genomic DNA was purified from TZM-bl cells using the Gentra Puregene Tissue Kit (QIAGEN, catalog #158667; input: 1-2×10⁷ cells) and sheared using the Covaris S220 acoustic sonicator (Woburn, MA) to an average length of 300 bp according to the manufacturer's protocol. The CIRCLE-Seq protocol was performed largely as previously reported. Briefly, sheared genomic DNA was subjected to solid phase reversible immobilization beads (SPRI) using Ampure XP Bead-based double size selection (Beckman Coulter, Jersey City, NJ; catalog #NC9959336, size range: 200-700 bp), end-repaired, A-tailed, and ligated (KAPA Biosystems, Wilmington, MA, catalog #KK8235) to a hairpin adapter (oSQT1288; 5′-P-CGGTGGACCGATGATCUATCGGTCCACCG*T-3′, where * indicates phosphorothioate linkage). The ligated, hairpin DNA fragments were treated with a mixture of Lambda Exonuclease and E. coli Exonulcease I (NEB catalog #M0262L, M0293L) to remove DNA with free ends. Next, adapter-ligated DNA was treated with USER enzyme (NEB, catalog #M5505L) and T4 polynucleotide kinase (NEB, catalog #M0201L), generating complementary 3′ overhangs to promote self-ligation and circularization of the DNA fragments. Resulting DNA (500 ng) was circularized overnight with T4 DNA ligase (NEB, catalog #M0202L) and was then treated with Plasmid-Safe ATP-dependent DNase (Epicentre, Madison, WI, catalog #E3101K) to remove non-circular DNA fragments before in vitro digestion with gRNA/SpCas9 nuclease (NEB, catalog M0386S). Cas9 treated DNA was A-tailed, ligated to the NEBNext adaptor for Illumina (NEB catalog #E7601_{A}), USER enzyme-treated, and amplified by PCR using KAPA Hifi polymerase (KAPA Biosystems, KK2601) and NEBNext® Multiplex Oligos for Illumina® (catalog #E7600S). Amplified DNA was subjected to another round of Ampure XP Bead-based double-sided size selection.

Completed DNA libraries were quantified by qPCR using the KAPA Library Quant Kit (KAPA Biosystems, catalog #07960140001), Qubit dsDNA quantitation, and sizing on an Agilent Fragment Analyzer prior to subsequent sequencing on an Illumina NextSeq500 instrument in the Genomics and Molecular Biology Shared Resource at the Dartmouth-Hitchcock Medical Center Core Facility (Lebanon, NH).

vii. CIRCLE-Seq Data Analysis

Completed DNA libraries were normalized, denatured and loaded onto flow cells and sequenced with 150 base pair paired end reads on the Illumina NextSeq500 instrument, with approximately 5 million sequence reads pairs per sample. A modified CIRCLE-Seq pipeline was implemented locally on a 12-Core iMac Pro. Briefly, de-multiplexed, trimmed, merged, paired end reads were mapped to a custom genome assembly comprised of the Human reference genome GRCh37 and HIV-1 HXB2 as a separate chromosome. Matched and unmatched sites were identified using default settings as previously published. In brief, read sequences with less than or equal to 6 nucleotide mismatches (including deletions and insertion) to the target (guide) +PAM sequence were identified as off-target sites, while those with greater than 6 nucleotide mismatches were categorized as unmatched.

3. Results

i. In Vitro Cleavage of the HIV-1 Proviral LTR Sequence

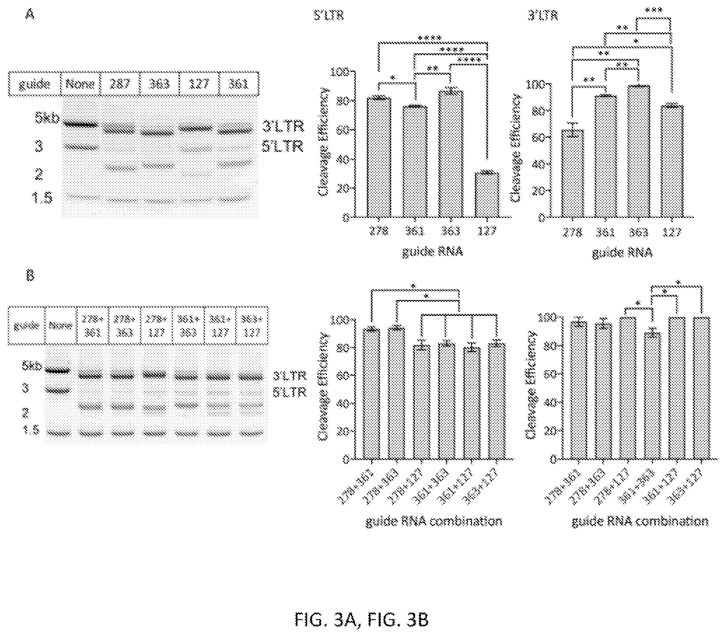

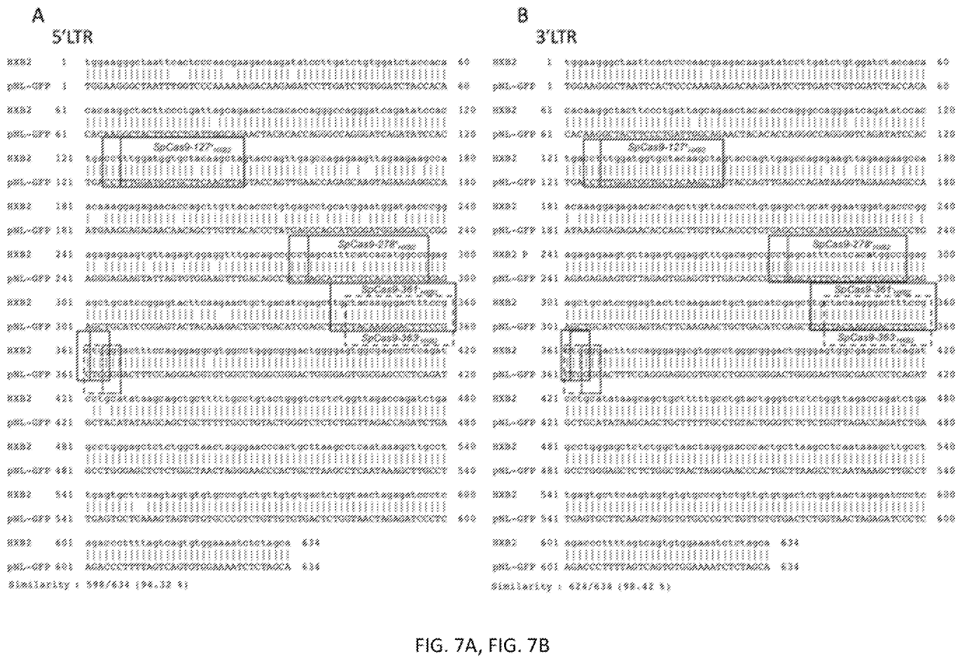

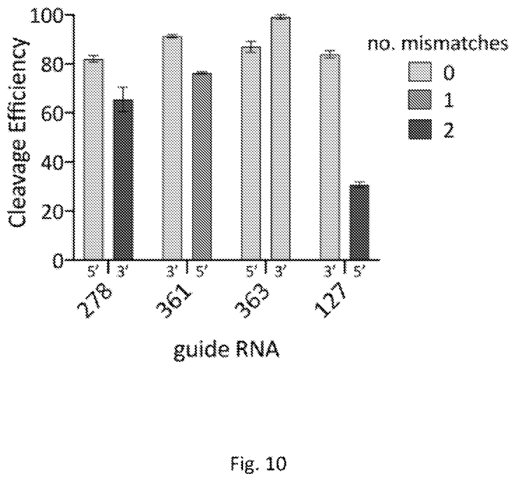

In vitro cleavage assays were performed to test the specificity and cleavage activity of various gRNAs targeting the HIV-1 LTR sequences present in the pNL-GFP plasmid. Individual gRNAs were complexed with SpCas9 tracrRNA and were combined with recombinant SpCas9 to form RNPs. RNPs were then incubated with restriction enzyme-digested pNL-GFP. Cleavage efficiencies were assessed by monitoring the production and intensity of expected cleavage products visualized by agarose gel electrophoresis. FIG. 8, and FIG. 3A, left panel, depict agarose gel images of target DNA cleavage. The percentage of target region cleaved is depicted in bar graph format (FIG. 3A, middle and right panels) and shows that the gRNAs SpCas9-363-HXB2 (363) and SpCas9-361-HXB2 (361) were the most effective in cleaving both the 5′ and 3′ LTRs, whereas SpCas9-278+HXB2 (278) and SpCas9-127+HXB2 (127) showed disparate cleavage efficiencies of the 5′ and 3′ LTR regions. It is likely that differences in cleavage efficiencies between the 5′ and 3′ LTRs could be attributed to differences in gene sequences at these regions in our target (FIG. 7, FIG. 10), although it cannot be ruled out that various gRNA-containing RNPs are inherently more efficient at cleaving target DNA.

CRISPR/Cas9 RNPs targeting different regions of the HIV-1 LTR were more efficient at gene cleavage when used in combination compared to single RNPs. As shown in FIG. 3B, the use of combinations of gRNAs demonstrated similar or higher cleavage efficiencies when compared to individual gRNAs. For example, the combination of gRNA 278 plus gRNA 361 (93.4±0.6% cleavage), and gRNA 278 plus gRNA 363 (94.3±0.8% cleavage) showed significantly higher target region cleavage at the 5′LTR compared to other combinations we tested. In contrast, when quantifying cleavage at the 3′LTR with combinations of gRNAs, all gRNA combinations showed increased cleavage efficiency compared to single gRNAs. The two best guide combinations, gRNA 278 plus 361 (96.8±3.2%), and gRNA 278 plus 363 (95.6±3.4%), had slightly increased cleavage efficiency in the 3′LTR compared to the 5′LTR. This pattern of differential cleavage to the 5′ and 3′ LTRs was consistent for both individual gRNAs as well as combinations of two different gRNAs used simultaneously (cleavage efficiencies ranged from 80% to 100%).

ii. Guide RNAs to HIV-1 LTRs Target HIV-1 Clades with Variable Efficiencies

While the activity of each guide RNA against a single HIV-1 clade gives some indication of functional activity, the ability to target various LTR sequences should also be assessed. Therefore, plasmids containing divergent portions of the 3′ LTRs from HIV-1 clades A through G were used in an in vitro assay similar to that performed above. gRNA 127 was tested because this gRNA had the greatest homology to the target region among all the various clades, as well as gRNA 363 that had the least homology across all of the 3′LTRs of the various clades.

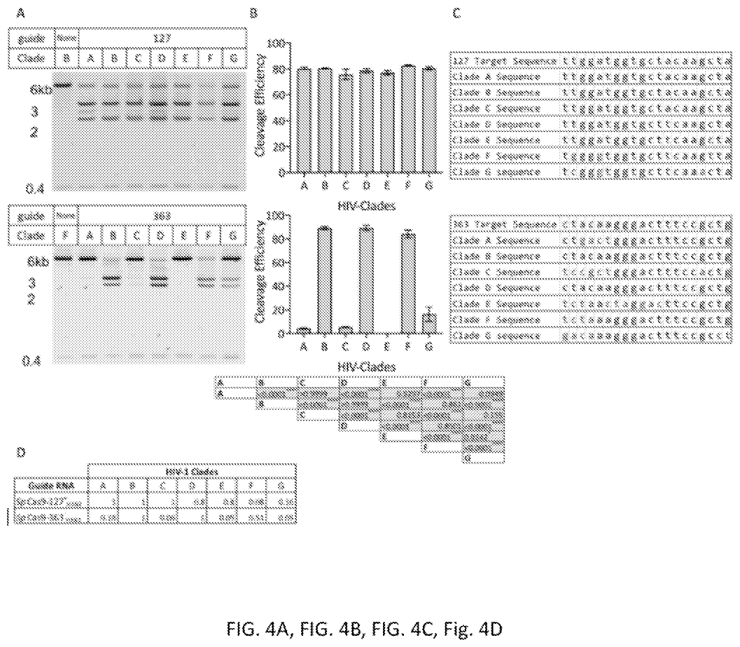

When testing in vitro cleavage, the cleavage efficiencies of these gRNAs correlated with similarities in homology between the target sequence and the guide RNAs among the various HIV clades A-G (FIG. 4, panels A-C). Target cleavage was assessed by agarose gel electrophoresis (FIG. 4A) and the percent cleavage efficiency was calculated and is shown in FIG. 4B. FIG. 4C shows the nucleotide mismatches between gRNA and target DNA, which in some cases were non-contiguous and located both proximal as well as distal to the PAM. Cutting Frequency Determination (CFD) scores were calculated for each guide using a pairwise comparison with each representative clade sequence to predict the impact of mismatches on cleavage efficiencies (FIG. 4D). This analysis considers the number of mismatches between guide RNA and target DNA, the location(s) of the mismatches, the PAM, and the specific nucleotide mismatch. Cleavage efficiencies for guide RNA 127 ranged from 76±4% to 83 0.5%, and were similar among clades (FIG. 4B). In contrast, RNPs with gRNA 363, which had a greater degree of nucleotide mismatch with the 3′ LTR, showed reduced target gene cleavage (FIG. 4B). Matches between gRNA 363 and target sequences (clade B and D) resulted in the highest cleavage efficiency (89±1.3% and 89±2.3%, respectively) (FIG. 4B). The sequence targeted by gRNA 363 contained 4 nucleotide mismatches with the corresponding sequence in HIV-1 clades A and G, and these mismatches were clustered proximal to the PAM (FIG. 4C). The degree of mismatch corresponded to decreases in cleavage efficiency for clade A (4.2±0.6%) and for clade G (16±6.1%) (FIG. 4B). There were six base pair mismatches between gRNA 363 and the 3′ LTR of clade C, and consequently demonstrated very low levels of cleavage of this target (5.4±0.3%) (FIGS. 4B & 4C). There were 10 base pair mismatches between gRNA 363 and clade E, and this resulted in a complete lack of target cleavage (FIG. 4B). In contrast to this pattern, gRNA 363 had 4 nucleotide mismatches with the 3′ LTR of Clade F, but demonstrated high levels of target cleavage (84±3%) (FIG. 4B). These results show the impact of sequence diversity on the potential therapeutic utility of anti-HIV-1 gRNAs, and also show that imperfect complementarity between target and gRNA does not prohibit broad use of a given gRNA across HIV-1 sequences.

iii. In Vivo Cleavage of HIV-1 Proviral 5′ LTR in TZM-Bl Cells

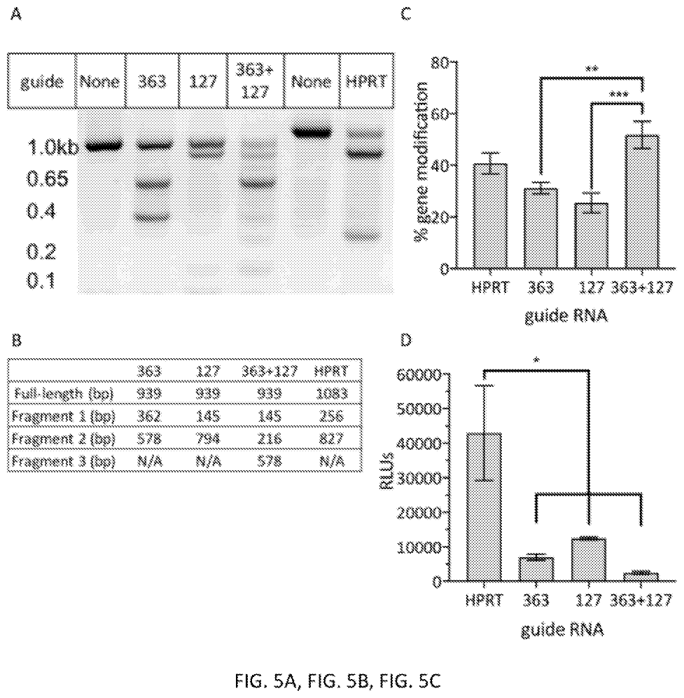

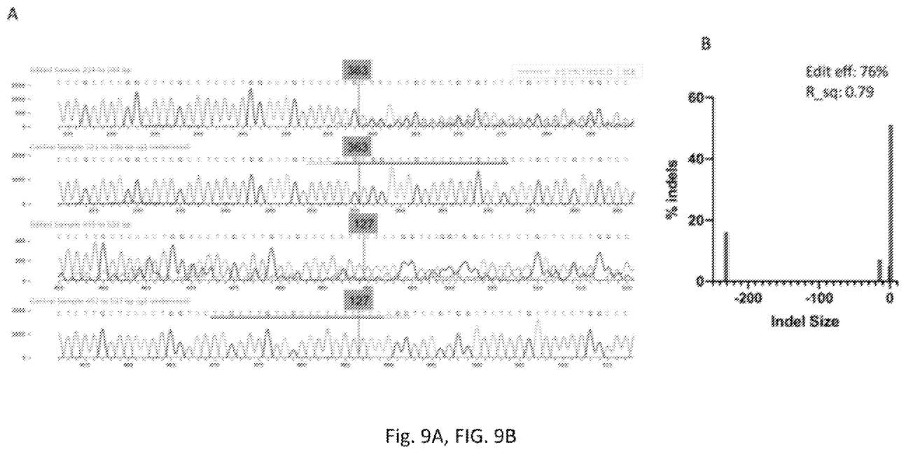

To assess the efficacy of anti-HIV-1 gRNAs following delivery to cells, in vivo assays in TZM-bl cells that contain integrated copies of two modified forms of the HIV-1 provirus were performed. Four days after transfection with RNPs, the genomic DNA was isolated from the cells, and the percentage of gene modification resulting from cleavage of the 5′ LTR was determined using PCR followed by the T7E1 assay. An optimized control gRNA targeting the hypoxanthine phosphoribosyl transferase (HPRT) gene was used as a positive control. The percent gene modification following transfection of RNPs targeting the HPRT gene was 41±4.0% (FIGS. 5A and 5C). The expected fragment sizes following cleavage with each guide is shown in FIG. 5B. Transfection with RNPs expressing gRNA 363 or gRNA 127 resulted in similar frequencies of modification of the 5′ LTR compared to those using the HPRT guide RNA. The co-transfection of RNPs containing gRNA 363 and gRNA 127 demonstrated the highest levels of gene modification (52±5.2%) and was significantly higher than transfection of either gRNA alone (31±2.2% gene modification with gRNA 363, and 25±3.9% gene modification with gRNA 127) (FIG. 5C). Furthermore, use of the two different guide RNAs resulted in a 231 base pair deletion event (FIG. 9). Given that the deletion event is not quantified by the T7E1 assay, it is possible that the targeting of the 5′ LTR by transfection of both gRNA 363 and gRNA 127 was higher than what was measured. A luciferase reporter assay was performed to examine functional activity of the LTR after transfection with the RNPs stated above. Cells treated with LTR guide RNPs demonstrated significantly less luciferase expression than those treated with the HPRT RNP. Guide RNAs 363 and 127, either alone or in combination, mediated functional damage of the LTR, damaging transcriptional capability.

iv. CIRCLE Seq Analysis of On- and Off-Target Cleavage Events

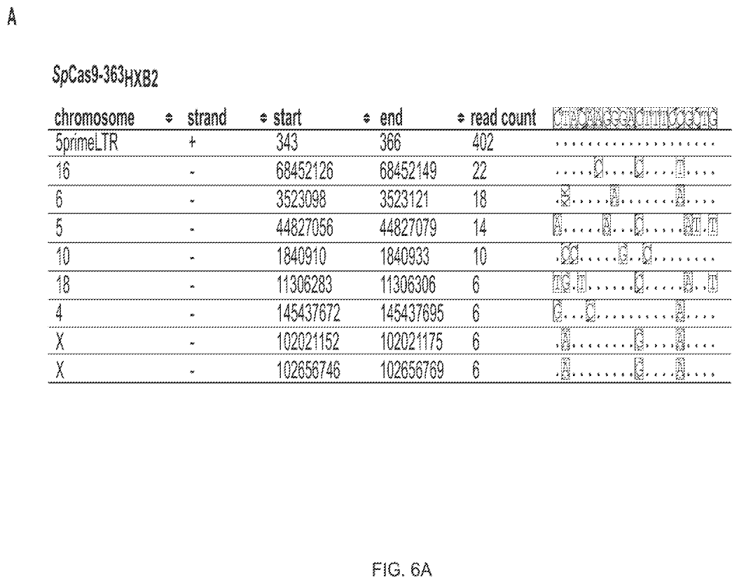

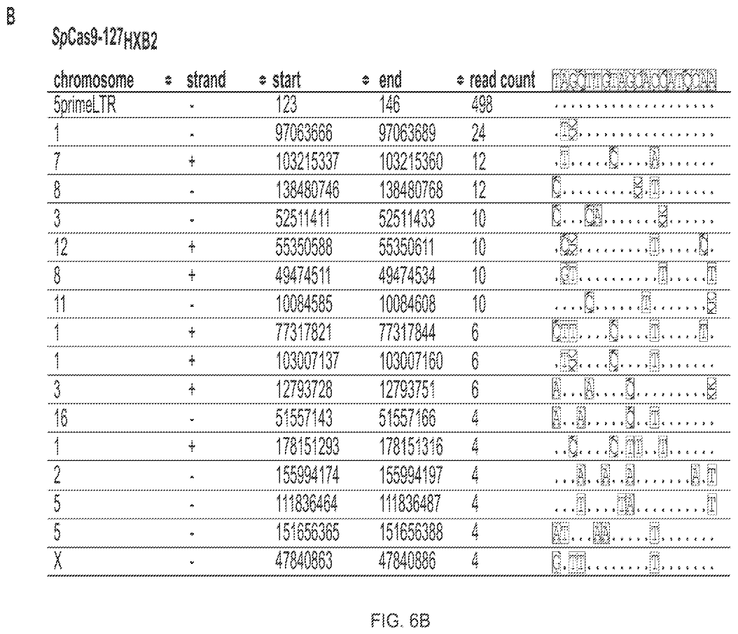

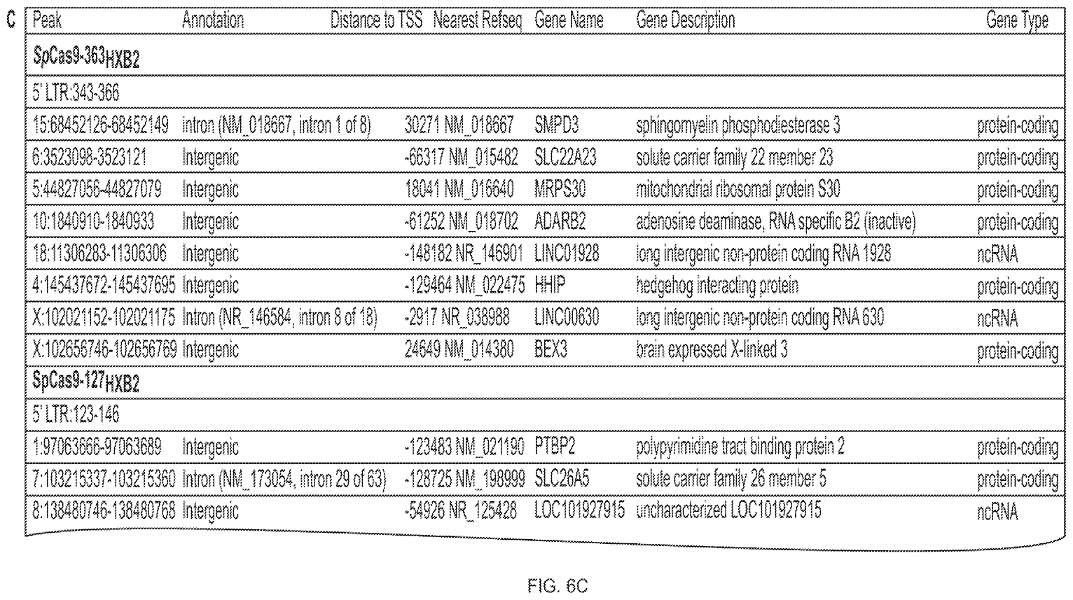

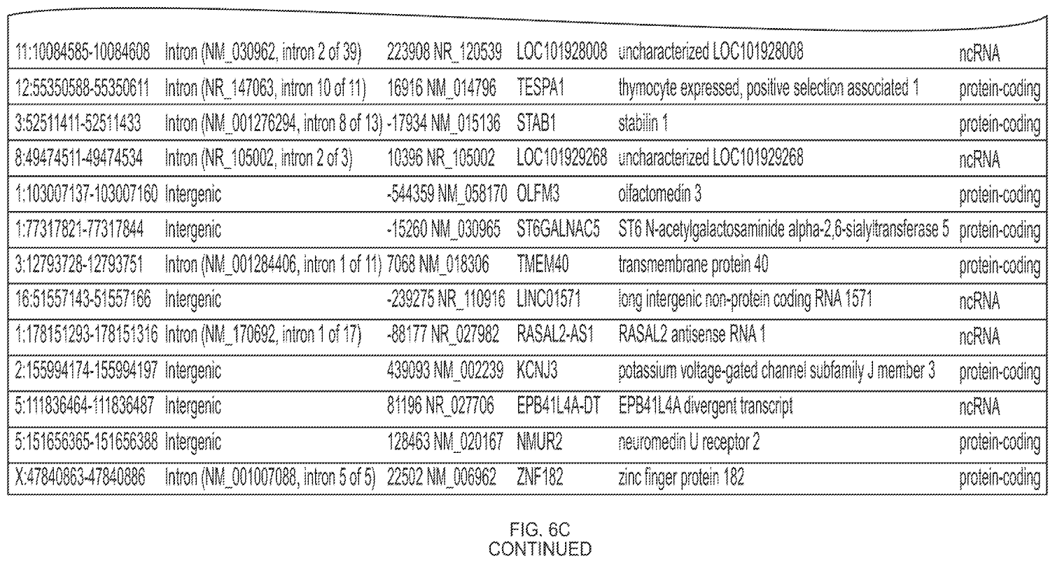

CIRCLE-Seq was performed to assess off-target cleavage events resulting from the use of several different gRNAs. To perform CIRCLE-Seq, circularized genomic DNA from the TZM-bl cell line was used as a target for in vitro cleavage using RNPs consisting of SpCas9, tracrRNA and either gRNA 127 or gRNA 363. After adaptor ligation and library preparation, off-target events induced by CRISPR/Cas9 cleavage were assessed by next generation sequencing. The most common off-target cleavage events for these gRNAs occurred at approximately 5% of the frequency of on-target cleavage events (FIGS. 6A & 6B). This was a relatively high degree of target specificity relative to other published guides tested by CIRCLE-Seq [14]. An analysis was also performed to identify the nearest annotated gene, and to delineate the position of the predicted off-target cleavage site relative to the target gene (FIG. 6C). While several intronic cleavage events were detected, there was no detection of any off-target events within known protein coding sequences. These data provide a list of potential off-target events that could be assessed by targeted, capture, or amplicon-based resequencing during preclinical development of identified gRNAs. Taken together, these data show the design and implementation of gRNAs capable of effectively and specifically targeting the LTRs of HIV-1 of multiple clades, and provide a framework for the development and thorough assessment of gRNA candidates.

4. Discussion

The development of CRISPR/Cas gene editing to cleave the integrated HIV-1 provirus and prevent new virus production provides new opportunities for the development of innovative therapeutic approaches. This method requires the design of guide RNA molecules that bind to a small region within the target DNA sequence, and directs the double-stranded DNA cleavage event by the Cas9 endonuclease. When over 1,200 HIV-1 LTR sequences were aligned to more than 500 potential guide RNA sites, the most conserved target regions were found in 70% of sequences studied, including all main M group subtypes. In another analysis, guides targeting within or proximal to the TAR encoding region were predicted to cleave 100% of clade B sequences, and 96.1% of unique sequences from each common subtype.

Single edits by CRISPR/Cas could eventually lead to viral escape, and thus the complete excision of the viral genome using gRNAs to the LTRs would avoid the potential for viral escape. In addition, targeting highly conserved regions in the genome was also found to decrease the chance of viral escape because the most conserved regions are essential for viral integrity and are less tolerable to mutations. Alternatively, the use of more than one gRNA (multiplexing) to target two distinct regions in the viral genome at the same time has also been found to significantly decrease viral escape following gene editing.

As shown herein, gRNA/Cas9 pairs exhibited significant cleavage activity on sequences with as many as four mismatches. CIRCLE-seq analyses also detected cleavage events at sites with DNA or RNA bulges. Allowing for single RNA or DNA bulges and four bases of misalignment, results in the identification of more than three thousand off-target sites using in silico tools such as Cas-OFFinder. Recently, methods for in vitro identification of Cas cleavage sites have emerged as an alternative or supplement to in silico prediction. These methods use genomic DNA and in vitro cleavage reactions with gRNA/Cas9 endonucleases to identify the universe of preferred cleavage sites. Two of these methods, CIRCLE-Seq and SITE-Seq, have been coupled to amplicon based-sequencing to allow detailed examination of Cas9-mediated on and off-target cleavage events in cells.

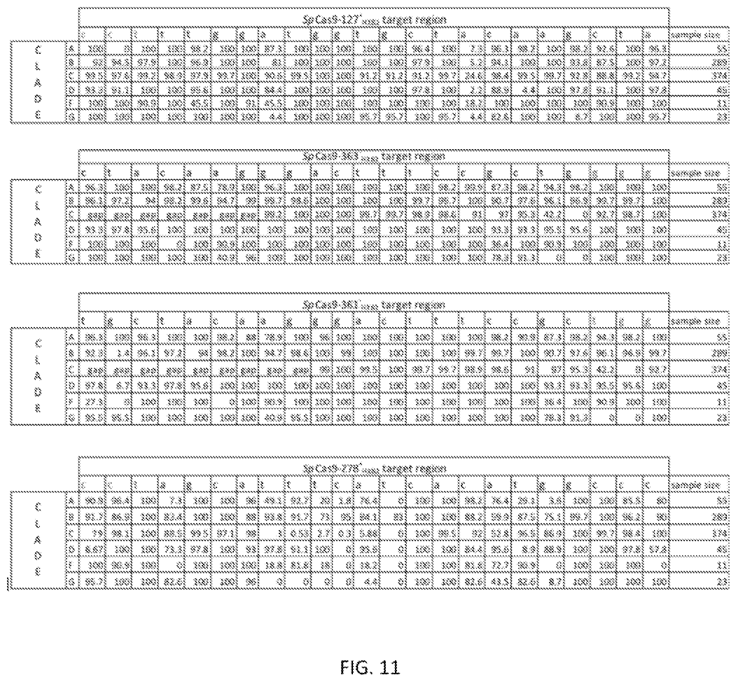

The design and testing of several guide RNAs that target the 5′ and 3′ long-terminal repeat (LTR) region of the HIV-1 provirus are described herein. Ribonucleoproteins (RNPs) containing a gRNA/tracrRNA and SpCas9 were prepared, and these complexes were used to achieve in vitro modification of target DNA, as well as in vivo delivery to assess the cleavage efficiency of the guide RNAs in nucleated cells. Significant cleavage efficiencies of the target DNA were observed in vitro. In vivo cleavage efficiencies reached as high as 50% and correlated with functional damage to the LTR by reductions in luciferase activity in the TZM-bl cell line. Other methods, in addition to the T7 endonuclease 1 mutation detection (T7E1) assay used to quantify gene cleavage in vivo, include interference of cleavage edits (ICE), and tracking of indels by decomposition (TIDE). These studies were followed by an in-depth analysis of the on-target and off-target efficiencies of the gRNAs against the target sequence. These findings show that the guide RNAs designed had high levels of cleavage efficiencies when used individually, and in some cases, the use of two guides increased the proportion of target cleavage over a single guide. It was also determined that gRNA 127 showed high levels of cleavage of different LTR sequences from HIV-1 clades A to G indicating that efficient gene cleavage can occur even with less than perfect homology between the guide RNA and target. These results are consistent with previous analyses of off-target cleavage events in vitro. To assess the conservation of guide RNA target regions across the different HIV-1 clade sequences at the 3′ LTR, each of four different gRNAs were aligned to their target sequence in each of the eight clades. Then the frequency by which the guide RNA matched each clade was assessed by comparing to multiple isolates of HIV within each clade. These analyses showed a conservation of target region across clades, and within a high percentage of different isolates from each clade (FIG. 11).

These studies highlight several considerations in the use of CRISPR/Cas9 for eliminating viral targets, including HIV-1.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. de Buhr, H. and R. J. Lebbink, Harnessing CRISPR to combat human viral infections. Curr Opin Immunol, 2018. 54: p. 123-129.
2. Hu, W., et al., RNA-directed gene editing specifically eradicates latent and prevents new HIV-1 infection. Proc Natl Acad Sci USA, 2014. 111(31): p. 11461-6.
3. Jinek, M., et al., A programmable dual-RNA-guided DNA endonuclease in adaptive bacterial immunity. Science, 2012. 337(6096): p. 816-21.
4. Lin, S. R., et al., The CRISPR/Cas9 System Facilitates Clearance of the Intrahepatic HBV Templates In Vivo. Mol Ther Nucleic Acids, 2014. 3: p. e186.
5. Wang, W., et al., CCR5 Gene Disruption via Lentiviral Vectors Expressing Cas9 and Single Guided RNA Renders Cells Resistant to HIV-1 Infection. PLoS One, 2014. 9(12): p. el 15987.
6. Kang, H., et al., CCR5 Disruption in Induced Pluripotent Stem Cells Using CRISPR/Cas9 Provides Selective Resistance of Immune Cells to CCR5-tropic HIV-1 Virus. Mol Ther Nucleic Acids, 2015. 4: p. e268.
7. Liu, Z., et al., Genome editing of the HIV co-receptors CCR5 and CXCR4 by CRISPR-Cas9 protects CD4(+) T cells from HIV-1 infection. Cell Biosci, 2017. 7: p. 47.
8. Xu, L., et al., CRISPR/Cas9-Mediated CCR5 Ablation in Human Hematopoietic Stem/Progenitor Cells Confers HIV-1 Resistance In Vivo. Mol Ther, 2017. 25(8): p. 1782-1789.
9. Panfil, A. R., et al., CRISPR/Cas9 Genome Editing to Disable the Latent HIV-1 Provirus. Front Microbiol, 2018. 9: p. 3107.
10. Xiao, Q., D. Guo, and S. Chen, Application of CRISPR/Cas9-Based Gene Editing in HIV-1/AIDS Therapy. Front Cell Infect Microbiol, 2019. 9: p. 69.
11. https://www.hiv.lanl.gov/content/sequence/HIV/CRFs/CRFs.html.
12. Bbosa, N., P. Kaleebu, and D. Ssemwanga, HIV subtype diversity worldwide. Curr Opin HIV AIDS, 2019. 14(3): p. 153-160.
13. Hemelaar, J., et al., Global and regional molecular epidemiology of HIV-1, 1990-2015: a systematic review, global survey, and trend analysis. The Lancet Infectious Diseases, 2019. 19(2): p. 143-55.
14. Tsai, S. Q., et al., CIRCLE-seq: a highly sensitive in vitro screen for genome-wide CRISPR-Cas9 nuclease off-targets. Nat Methods, 2017. 14(6): p. 607-614.
15. Adachi, A., et al., Production of acquired immunodeficiency syndrome-associated retrovirus in human and nonhuman cells transfected with an infectious molecular clone. J Virol, 1986. 59: p. 284-291.
16. http://blast.ncbi.nlm.nih.gov/Blast.cgi.
17. Gibson, D. G., et al., Enzymatic assembly of DNA molecules up to several hundred kilobases. Nat Methods, 2009. 6(5): p. 343-5.
18. Gibson, D. G., Enzymatic assembly of overlapping DNA fragments. Methods Enzymol, 2011. 498: p. 349-61.
19. Mali, P., et al., RNA-Guided Human Genome Engineering via Cas9. Science, 2013. 339(6121): p. 823-6.
20. https://www.idtdna.com/site/order/designtool/index/CRISPR_CUSTOM.
21. Jeeninga, R. E., et al., Functional differences between the long terminal repeat transcriptional promoters of human immunodeficiency virus type 1 subtypes A through G. J Virol, 2000. 74(8): p. 3740-51.
22. Klaver, B. and B. Berkhout, Comparison of 5′ and 3′ long terminal repeat promoter function in human immunodeficiency virus. J Virol, 1994. 68(6): p. 3830-40.
23. Platt, E. J., et al., Evidence that ecotropic murine leukemia virus contamination in TZM-bl cells does not affect the outcome of neutralizing antibody assays with human immunodeficiency virus type 1. J Virol, 2009. 83(16): p. 8289-92.
24. Platt, E. J., et al., Effects of CCR5 and CD4 cell surface concentrations on infections by macrophagetropic isolates of human immunodeficiency virus type 1. J Virol, 1998. 72(4): p. 2855-64.
25. Takeuchi, Y., M. O. McClure, and M. Pizzato, Identification of gammaretroviruses constitutively released from cell lines used for human immunodeficiency virus research. J Virol, 2008. 82(24): p. 12585-8.
26. Wei, X., et al., Emergence of resistant human immunodeficiency virus type 1 in patients receiving fusion inhibitor (T-20) monotherapy. Antimicrob Agents Chemother, 2002. 46(6): p. 1896-905.
27. Derdeyn, C. A., et al., Sensitivity of human immunodeficiency virus type 1 to the fusion inhibitor T-20 is modulated by coreceptor specificity defined by the V3 loop of gp120. J Virol, 2000. 74(18): p. 8358-67.
28. Guschin, D. Y., et al., A rapid and general assay for monitoring endogenous gene modification. Methods Mol Biol, 2010. 649: p. 247-56.
29. https://github.com/tsailabSJ/circleseq.
30. Doench, J. G., et al., Optimized sgRNA design to maximize activity and minimize off-target effects of CRISPR-Cas9. Nat Biotechnol, 2016. 34(2): p. 184-191.
31. Geonnotti, A. R., et al., Differential inhibition of human immunodeficiency virus type 1 in peripheral blood mononuclear cells and TZM-bl cells by endotoxin-mediated chemokine and gamma interferon production. AIDS Res Hum Retroviruses, 2010. 26(3): p. 279-91.
32. Sullivan, N. T., et al., Novel gRNA design pipeline to develop broad-spectrum CRISPR/Cas9 gRNAs for safe targeting of the HIV-1 quasispecies in patients. Sci Rep, 2019. 9(1): p. 17088.
33. Roychoudhury, P., et al., Viral diversity is an obligate consideration in CRISPR/Cas9 designs for targeting the HIV reservoir. BMC Biol, 2018. 16(1): p. 75.
34. Dampier, W., et al., Designing broad-spectrum anti-HIV-1 gRNAs to target patient-derived variants. Sci Rep, 2017. 7(1): p. 14413.
35. Hsu, P. D., et al., DNA targeting specificity of RNA-guided Cas9 nucleases. Nat Biotechnol, 2013. 31(9): p. 827-32.
36. Wang, Z., et al., HIV-1 Employs Multiple Mechanisms To Resist Cas9/Single Guide RNA Targeting the Viral Primer Binding Site. J Virol, 2018. 92(20).
37. Das, A. T., C. S. Binda, and B. Berkhout, Elimination of infectious HIV DNA by CRISPR-Cas9. Curr Opin Virol, 2019. 38: p. 81-88.
38. Lebbink, R. J., et al., A combinational CRISPR/Cas9 gene-editing approach can halt HIV replication and prevent viral escape. Sci Rep, 2017. 7: p. 41968.
39. Darcis, G., et al., The Impact of HIV-1 Genetic Diversity on CRISPR-Cas9 Antiviral Activity and Viral Escape. Viruses, 2019. 11(3).
40. Wang, G., et al., A Combinatorial CRISPR-Cas9 Attack on HIV-1 DNA Extinguishes All Infectious Provirus in Infected T Cell Cultures. Cell Rep, 2016. 17(11): p. 2819-2826.
41. Jamal, M., et al., Improving CRISPR-Cas9 On-Target Specificity. Curr Issues Mol Biol, 2018. 26: p. 65-80.
42. Zischewski, J., R. Fischer, and L. Bortesi, Detection of on-target and off-target mutations generated by CRISPR/Cas9 and other sequence-specific nucleases. Biotechnol Adv, 2017. 35(1): p. 95-104.
43. Akcakaya, P., et al., In vivo CRISPR editing with no detectable genome-wide off-target mutations. Nature, 2018. 561(7723): p. 416-419.
44. Bae, S., J. Park, and J. S. Kim, Cas-OFFinder: a fast and versatile algorithm that searches for potential off-target sites of Cas9 RNA-guided endonucleases. Bioinformatics, 2014. 30(10): p. 1473-5.
45. Cameron, P., et al., Mapping the genomic landscape of CRISPR-Cas9 cleavage. Nat Methods, 2017. 14(6): p. 600-606.
46. Hadden, J. M., et al., Crystal structure of the Holliday junction resolving enzyme T7 endonuclease I. Nature Structural Biology, 2001. 8(1): p. 62-67.
47. Hsiau, T., et al., Inference of CRISPR Edits from Sanger Trace Data. https://www.biorxiv.org/content/biorxiv/early/2019/01/14/251082.full.pdf.
48. Brinkman, E. K., et al., Easy quantitative assessment of genome editing by sequence trace decomposition. Nucleic Acids Res, 2014. 42(22): p. e168.

We claim:

1. A guide RNA (gRNA) that specifically binds a 5′ LTR human immunodeficiency virus-1 (HIV-1) sequence comprising TTGGATGGTGCTTCAAGTTA (SEQ ID NO:1).

2. A gRNA that specifically binds a 5′ LTR HIV-1 sequence comprising
(SEQ ID NO: 2)
CTACAAGGGACTTTCCGCTG.

3. A gRNA that specifically binds a 5′ LTR HIV-1 sequence comprising
(SEQ ID NO: 3)
TCTACAAGGGACTTTCCGCT.

4. The gRNA of any one of claims 1-4, further comprising a nucleic acid sequence that binds a Cas protein.

5. A nucleic acid sequence comprising a nucleic acid sequence encoding one or more gRNAs, wherein said one or more gRNAs hybridizes with a target sequence in HIV-1, wherein the target sequence is selected from the group consisting of SEQ ID NO:1 SEQ ID NO:2, and SEQ ID NO:3.

6. The nucleic acid sequence of claim 5, further comprising a nucleic acid sequence encoding a cas protein.

7. A vector comprising a nucleic acid sequence encoding one or more gRNAs, wherein the one or more gRNA hybridizes with a target sequence in HIV-1, wherein the target sequence is selected from the group consisting of SEQ ID NO:1, SEQ ID NO:2, and SEQ ID NO:3.

8. The vector of claim 7, wherein the vector is an expression vector.

9. The vector of claim 8, wherein the expression vector is a viral vector.

10. The vector of claim 9, wherein the viral vector is a lentiviral vector.

11. The vector of any one of claims 7-10, wherein the vector further comprises a nucleic acid sequence encoding a cas protein.

12. A method for inhibiting the function of a target HIV-1 DNA sequence in a cell comprising contacting a cell comprising a cellular genome and harboring a HIV-1 genome comprising a target HIV-1 DNA sequence integrated into the cellular genome with
one or more gRNAs, or nucleic acids encoding said one or more gRNAs, and
a Clustered Regularly Interspaced Short Palindromic Repeats-Associated (cas) protein, or nucleic acid sequence encoding a cas protein,
wherein the one or more gRNAs uniquely hybridizes with the target HIV-1 DNA sequence, wherein the target HIV-1 DNA sequence is selected from the group consisting of SEQ ID NO:1, SEQ ID NO:2, and SEQ ID NO:3;
thereby inhibiting the function or presence of the target HIV-1 DNA sequence.

13. A method for removing a target HIV-1 DNA sequence from a cellular genome comprising contacting a cell comprising a cellular genome and harboring a HIV-1 genome comprising a target HIV-1 DNA sequence integrated into the cellular genome with
one or more gRNAs, or nucleic acids encoding said one or more gRNAs, and
a Clustered Regularly Interspaced Short Palindromic Repeats-Associated (cas) protein, or nucleic acid sequence encoding a cas protein,
wherein the one or more gRNAs uniquely hybridizes with the target HIV-1 DNA sequence, wherein the target HIV-1 DNA sequence is selected from the group consisting of SEQ ID NO:1, SEQ ID NO:2, and SEQ ID NO:3;
thereby removing the target HIV-1 DNA sequence from the cellular genome.

14. The method of any one of claims 12-13, wherein the one or more gRNAs do not bind to the cellular genome.

15. The method of any one of claims 12-14, wherein the one or more gRNAs can target a LTR region of two or more HIV clades.

16. The method of any one of claims 12-15, wherein the one or more gRNAs hybridize to a target HIV-1 DNA sequence in the LTR region of two or more HIV clades.

17. The method of any one of claims 12-16, wherein the target HIV-1 DNA sequence is SEQ ID NO:1, and wherein the one or more guide RNA, or nucleic acids encoding the one or more guide RNA comprise the sequence of SEQ ID NO:1, or the complement thereof.

18. The method of any one of claims 12-17, wherein the target HIV-1 DNA sequence is SEQ ID NO:2, and wherein the one or more guide RNA, or nucleic acids encoding the one or more guide RNA comprise the sequence of SEQ ID NO:2, or the complement thereof.

19. The method of any one of claims 12-18, wherein the target HIV-1 DNA sequence is SEQ ID NO:3, and wherein the one or more guide RNA, or nucleic acids encoding the one or more guide RNA comprise the sequence of SEQ ID NO:3, or the complement thereof.

20. The method of any one of claims 12-19, wherein the one or more guide RNA and the cas protein form a complex inside the cell, and wherein the complex cuts the HIV-1 DNA sequence, thereby inhibiting the function or presence of the target HIV-1 DNA sequence

21. The method of claim 20, wherein the complex cuts the HIV-1 DNA sequence at the 5′LTR and the 3′LTR, thereby inhibiting the function or presence of the target HIV-1 DNA sequence.

22. The method of any one of claims 12-21, wherein the cas protein is cas9.

23. The method of any one of claims 12-22, wherein the cas protein has been codon-optimized for expression in human cells.

24. The method of any one of claims 12-23, wherein the cas protein further comprises a nuclear localization sequence.

25. The method of any one of claims 12-24, wherein the nucleic acids encoding the one or more guide RNA, and the nucleic acids encoding the cas protein are contained in an expression vector.

26. The method of claim 25, wherein the expression vector is a viral vector.

27. The method of any one of claims 12-26, wherein contacting comprises contacting the cell with one or more expression vectors comprising the nucleic acids encoding the one or more guide RNA and the nucleic acids encoding the cas protein.

28. The method of any one of claims 12-27, wherein the contacting step is carried out in vitro.

29. The method of any one of claims 12-27, wherein the contacting step is carried out in vivo.

30. A kit, comprising:
one or more guide RNA, or nucleic acids encoding the one or more guide RNA, wherein the guide RNA hybridizes with a target HIV-1 DNA sequence; and
a cas protein, or a nucleic acid encoding the cas protein.

31. A set of vectors comprising:
a vector comprising a nucleic acid sequence encoding one or more gRNAs, wherein the one or more gRNA hybridizes with a target sequence in HIV-1, wherein the target sequence is selected from the group consisting of SEQ ID NO:1, SEQ ID NO:2, and SEQ ID NO:3; and
a vector comprising a nucleic acid sequence encoding a cas protein.
